(19) United States
(12) Patent Application Publication
(10) Pub. No.: US 2022/0152214 A1
(43) Pub. Date: May 19, 2022

(54) CANCER TREATMENT WITH ROR1 ANTIBODY IMMUNOCONJUGATES

(71) Applicant: VelosBio Inc., Rahway, NJ (US)

(72) Inventors: Langdon Miller, Washington, CA (US); Brian Lannutti, Solana Beach, CA (US); Katti Jessen, San Diego, CA (US)

(73) Assignee: VelosBio Inc., Rahway, NJ (US)

(21) Appl. No.: 17/426,830

(22) PCT Filed: Feb. 1, 2020
(86) PCT No.: PCT/US2020/016301

§ 371 Date: Jul. 29, 2021

Related U.S. Application Data
(60) Provisional application No. 62/800,187, filed on Feb. 1, 2019.

(51) Int. Cl. A61K 47/68; C07K 16/28; A61P 35/02

(52) U.S. Cl. CPC A61K 47/6849; A61K 47/6803; A61P 35/02; C07K 16/2803; A61K 47/6867

(57) ABSTRACT

Provided herein are methods for treating a cancer patient with immunoconjugates comprising an anti-ROR1 antibody or an antigen-fragment fragment thereof and a drug moiety.

### CROSS-REFERENCE TO RELATED APPLICATION

This application claims priority from U.S. Provisional Patent Application 62/800,187, filed Feb. 1, 2019, whose disclosure is incorporated by reference herein in its entirety.

### SEQUENCE LISTING

The instant application contains a Sequence Listing that has been submitted electronically in ASCII format and is hereby incorporated by reference in its entirety. The electronic copy of the Sequence Listing, created on Jan. 29, 2020, is named 024651_WO003_SL.txt and is 9,833 bytes in size.

### BACKGROUND OF THE INVENTION

Hematological malignancies comprise diseases resulting from transformation events occurring in immune or hematopoietic organs. Lymphoid malignancies arise from the accumulation of monoclonal neoplastic lymphocytes in lymph nodes and organs such as blood, bone marrow, spleen, and liver. Variants of these cancers comprise non-Hodgkin lymphomas (NHLs), including chronic lymphocytic leukemia (CLL), small lymphocytic lymphoma (SLL), mantle cell lymphoma (MCL), follicular lymphoma (FL), marginal zone lymphoma (MZL), diffuse large B-cell lymphoma (DLBCL), Richter transformation lymphoma (RTL), Burkitt lymphoma (BL), lymphoplasmacytoid lymphoma (LPL), Waldenström macroglobulinemia (WM), acute lymphocytic leukemia (ALL), and several types of T cell lymphomas. Acute myeloid leukemia (AML) results from the accumulation of neoplastic myeloid blasts in the bone marrow, blood, central nervous system, and other organs.

Depending partially upon the cell of origin (B cell or T cell), patients with NHL may experience disabling constitutional symptoms, lymphadenopathy and organomegaly that can induce life-threatening organ dysfunction, myelosuppression and immunocompromise that can result in susceptibility to infection and bleeding, and/or cutaneous manifestation that can be painful, intensely pruritic, and disfiguring. Patients with LPL/WM have an overproduction of immunoglobulin (Ig) M-producing plasma cells and can develop plasma hyperviscosity due to the presence of this circulating monoclonal IgM protein (M-protein). For patients with ALL or AML, disruption of normal bone marrow function by an expanding clone of leukemic blasts leaves them prone to life-threatening infection and bleeding.

Treatments for these diseases are intended to induce tumor regression, delay tumor progression, control disease-related complications, and extend life. Patients are commonly given chemotherapeutic and/or immunotherapeutic agents. Front-line therapies can provide durable remissions. However, many patients will eventually experience disease relapse; further sequential therapies are used to try to control disease manifestations. Despite use of agents with differing mechanisms of action, progressive tumor resistance often develops. Patients with multiple relapsed progressive disease have poor prognosis and are likely to die of their cancers. Thus, novel mechanisms of action are needed to safely offer new treatment options for patients with hematological cancers that have become resistant to existing therapies.

Receptor tyrosine kinase-like orphan receptor 1 (ROR1) is a cell-surface protein that mediates signals from its ligand, the secreted glycoprotein Wnt5a. Consistent with its role in influencing the fate of stem cells during embryogenesis, ROR1 expression is observed on invasive malignancies that revert to an embryonic transcriptional program, but is not observed in normal adult tissues. ROR1 thus offers a favorable selectivity profile as a therapeutic target. ROR1 is commonly expressed on the malignant cells of patients with hematological cancers, and is also present on the cell surfaces of multiple solid tumors, where it appears to be a marker for cancer stem cells.

In view of the high unmet medical need in many patients with hematological and other cancers and the role of ROR1 in cancer, there is a need for new therapies that can improve outcomes for patients, including patients who do not respond to existing therapies, through targeting of ROR1.

### SUMMARY OF THE INVENTION

The present invention relates to a method of treating a cancer patient using an immunoconjugate having the structure shown below.

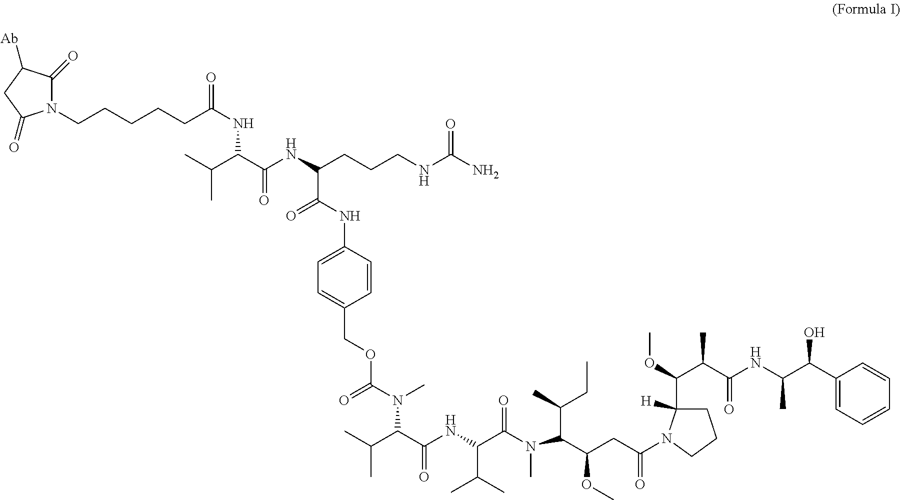

wherein Ab is an antibody that specifically binds to human receptor tyrosine kinase like orphan receptor 1 (ROR1), wherein the heavy chain and light chain of the antibody comprise the amino acid sequences of SEQ ID NOs: 1 and 2, respectively; and wherein the immunoconjugate is administered to the patient at a dose of 0.25 to 4.00 mg/kg. As used herein, Formula I above is not intended to denote that each Ab may be conjugated to only one copy of the drug moiety shown in the formula. In some embodiments, the number or copy of the drug moiety per antibody (DAR) ranges from 1 to 7, where each drug moiety is conjugated to the antibody through a linker as shown in Formula I.

In some embodiments, the immunoconjugate is administered (e.g., intravenously) according to a dosage regimen described herein. The immunoconjugate may be administered, for example, at a dose of 0.50, 0.75, 1.00, 1.25, 1.50, 1.75, 2.00, 2.25, 2.50, 2.75, or 3.00 mg/kg.

In certain embodiments, the immunoconjugate may be administered in repeated three-week cycles (e.g., on Day 1 or Days 1 and 8 per cycle). In certain embodiments, the immunoconjugate may be administered in repeated four-week cycles (e.g., on Days 1, 8, and 15 per cycle). In some embodiments, the number of cycles may total 3, 6, or more. In particular embodiments, the immunoconjugate may be administered: weekly for the first three, four, six, or eight weeks and then every three weeks; or every three weeks for the first three, six, or nine weeks and then weekly.

In some embodiments, the immunoconjugate is administered to a patient with a hematological cancer such as a lymphoid malignancy. In certain embodiments, the cancer is selected from the group consisting of CLL, SLL, MCL, FL, MZL, DLBCL, RTL, BL, LPL, WM, T cell NHL, ALL, and AML. In particular embodiments, the patient has been treated previously for the cancer, and/or has a cancer that is relapsed or refractory to treatment (e.g., one or more existing treatments for the cancer, such as all existing treatments).

In some embodiments, treatment with the immunoconjugate induces tumor regression (e.g., results in complete tumor eradication); delays tumor progression; inhibits cancer metastasis; prevents cancer recurrence or residual disease; decreases the size of nodal or extranodal tumor masses; decreases malignant cell numbers in bone marrow and peripheral blood; decreases malignant splenomegaly or hepatomegaly; improves cancer-related anemia, neutropenia, or thrombocytopenia; ameliorates cutaneous manifestation; decrease the likelihood of hyperviscosity syndrome in patients with LPL/WM; ameliorates disabling constitutional symptoms; and/or prolongs survival.

The present disclosure also provides an immunoconjugate for use in treating cancer in a patient in a method described herein. Further, the present disclosure provides the use of an immunoconjugate for the manufacture of a medicament for treating cancer in a patient in a method described herein.

### BRIEF DESCRIPTION OF THE DRAWINGS

FIG. 1 is a graph showing plasma concentration-time curves documenting total ADC-A (solid line) and MMAE (dotted line) plasma exposures. C: Cycle. D: Day. LLQ: Lower limit of quantification.

FIG. 2 is a set of graphs demonstrating ADC-A engagement of ROR1 on circulating leukemia cells (top panels) and ADC-A and MMAE plasma concentrations (bottom panels) over time in two subjects with CLL. Subject 1: left. Subject 2: right.

FIG. 3 is a graph showing the correlation of unoccupied ROR1 receptors with ADC-A plasma concentration.

FIG. 4 is a graph depicting pharmacokinetic simulations of ADC-A plasma concentrations over time with Q1/3W, Q2/3W, and Q3/4W dosing regimens.

FIG. 5 is a graph showing the best change in tumor dimensions by ADC-A starting dose. TE: too early to evaluate. PR: partial response.

### DETAILED DESCRIPTION OF THE INVENTION

The present invention provides treatment regimens using a ROR1 immunoconjugate. These treatment regimens may be used to treat a variety of cancers, such as those that are expected to express ROR1.

### 1. Immunoconjugates

An “antibody-drug conjugate,” or “ADC,” or “immunoconjugate,” refers to an antibody molecule or an antigen-binding fragment thereof that is covalently or non-covalently bonded, with or without a linker, to one or more biologically active molecule(s). The present immunoconjugates comprise antibodies or fragments thereof that are specific for human ROR1 and thus can serve as excellent targeting moieties for delivering the conjugated payloads to cells (e.g., ROR1-positive cells). In certain embodiments, an immunoconjugate used in a treatment regimen of the invention is an immunoconjugate described in WO 2018/237335.

Shown below are SEQ ID NOs for the amino acid sequences of the heavy and light chain complementarity-determining regions (HCDRs and LCDRs), heavy and light chain variable domains (VH and VL), and heavy and light chains (HC and LC) of an exemplary anti-ROR1 antibody used in the immunoconjugates described herein:

| HCDR1 | HCDR2 | HCDR3 | VH | HC | LCDR1 | LCDR2 | LCDR3 | YL | LC |
|---|---|---|---|---|---|---|---|---|---|
| 5 | 6 | 7 | 3 | 1 | 8 | 9 | 10 | 4 | 2 |

In some embodiments, the antibody or antibody fragment in the immunoconjugate specifically binds human ROR1, and its heavy and light chains respectively comprise:
a) the HCDR1-3 amino acid sequences in SEQ ID NO: 1, and the LCDR1-3 amino acid sequences in SEQ ID NO: 2;
b) HCDR1-3 comprising the amino acid sequences of SEQ ID NO: 5-7, respectively, and LCDR1-3 comprising the amino acid sequences of SEQ ID NOs: 8-10, respectively;
c) HCDR1-3 comprising residues 26-33, 51-58, and 97-105 of SEQ ID NO: 3, respectively, and LCDR1-3 comprising residues 27-32, 50-52, and 89-97 of SEQ ID NO: 4, respectively;
d) HCDR1-3 comprising residues 26-32, 52-57, and 99-105 of SEQ ID NO: 3, respectively, and LCDR1-3 comprising residues 24-34, 50-56, and 89-97 of SEQ ID NO: 4, respectively; or
e) HCDR1-3 comprising residues 31-35, 50-66, and 99-105 of SEQ ID NO: 3, respectively, and LCDR1-3 comprising residues 24-34, 50-56, and 89-97 of SEQ ID NO: 4, respectively.

In certain embodiments of an immunoconjugate described herein, the antibody can be conjugated to the cytotoxic agent via a linker. In some embodiments, the linker is a cleavable linker. A cleavable linker refers to a linker that comprises a cleavable moiety and is typically susceptible to cleavage under in vivo conditions. In exemplary embodiments, the linker may comprise a dipeptide, such as a valine-citrulline (val-cit or VC) linker. In certain embodiments, the linker is attached to a cysteine residue on the antibody.

In some embodiments, the conjugation of the linker/payload to the antibody or fragment may be formed through reaction with a maleimide group (which may also be referred to as a maleimide spacer). In certain embodiments, the maleimide group is maleimidocaproyl (mc); thus, the linker/payload is conjugated to the antibody or fragment through reaction between a residue on the antibody or fragment and the me group in the linker precursor.

In some embodiments, the linker may include a benzoic acid or benzyloxy group, or a derivative thereof. In some embodiments, the linker includes a para-amino-benzyloxycarbonyl (PAB) group.

In some embodiments, the linkage between the Ab and payload or drug (D) components of the immunoconjugate may be formed through reaction of the components with a linker comprising a maleimide group, a peptide moiety, and/or a benzoic acid (e.g., PAB) group, in any combination. In certain embodiments, the maleimide group is maleimidocaproyl (mc). In certain embodiments, the peptide group is Val-Cit (VC). In certain embodiments, the linker comprises a Val-Cit-PAB group. In certain embodiments, the conjugation of the linker to the antibody or fragment may be formed from an mc-Val-Cit group. In certain embodiments, the linkage between the antibody or fragment and the drug moiety may be formed from an mc-Val-Cit-PAB group.

Linkers can be conjugated to the anti-ROR1 antibodies and antigen-binding fragments of the current disclosure in multiple ways. Generally, a linker and a cytotoxic moiety are synthesized and conjugated before attachment to an antibody. One method of attaching a linker-drug conjugate to an antibody involves reduction of solvent-exposed disulfides with dithiothreitol (DTT) or tris (2-carboxyethyl)phosphine (TCEP), followed by modification of the resulting thiols with maleimide-containing linker-drug moieties (e.g., 6-maleimidocaproyl-valine-citrulline-p-aminobenzyloxycarbonyl (mc-VC-PAB)). A native antibody contains 4 inter-chain disulfide bonds and 12 intra-chain disulfide bonds, as well as unpaired cysteines. Thus, antibodies modified in this way can comprise greater than one linker-drug moiety per antibody. In certain embodiments, the immunoconjugates each comprise at least 1, 2, 3, 4, 5, 6, 7, 8, 9, or 10 linker/drug moieties. In certain embodiments, the immunoconjugates each comprise one or more (e.g., 1 to 10, 1 to 9, 1 to 8, 1 to 7, 1 to 6, 1 to 5, 1 to 4, 1 to 3, or 1 to 2) linker/drug moieties. In cases where the linker is branched and can each attach to multiple drug moieties, the ratio of the drug moiety to the antibody will be higher than using an unbranched linker.

In some embodiments, a suitable cytotoxic agent for use in an immunoconjugate described herein may be, e.g., an anti-tubulin agent such as an auristatin. In certain embodiments, the cytotoxic agent is monomethyl auristatin E (MMAE).

In some embodiments, an immunoconjugate described herein is constructed as follows:

| mAb | Conjugation | Linker Components | | Payload |
|---|---|---|---|---|
| anti-ROR1 antibody | MAL (mc) | VC | PAB | MMAE |

*MAL: maleimide chemistry (MAL).

The anti-ROR1 antibody may be an anti-ROR1 antibody described herein, e.g., an antibody with a heavy chain amino acid sequence of SEQ ID NO: 1 and a light chain amino acid sequence of SEQ ID NO: 2.

In particular embodiments, an immunoconjugate used in the treatment regimens of the invention has the following structure (I):

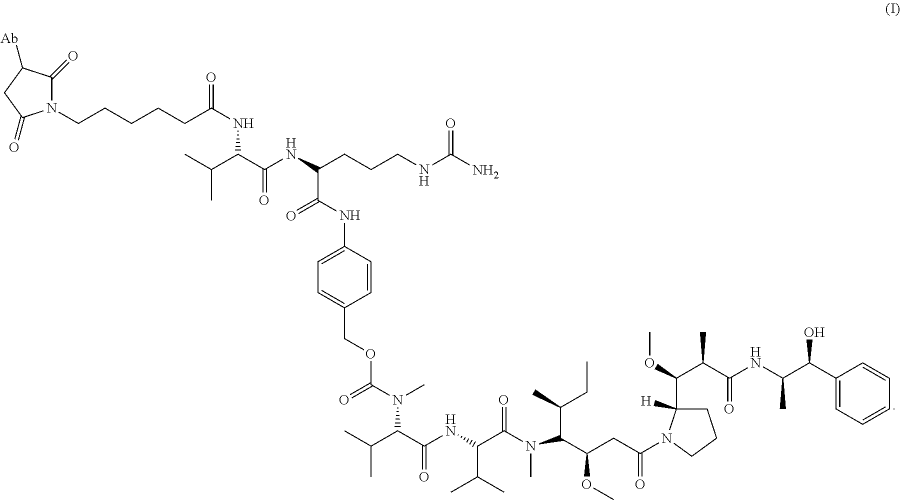

In some embodiments, the antibody is Ab1, which has a heavy chain amino acid sequence of SEQ ID NO: 1 and a light chain amino acid sequence of SEQ ID NO: 2 (Ab1); this immunoconjugate may have a DAR of 3-6 and is referred to herein as “ADC-A.” See also WO 2018/237335. The payload is conjugated to Ab1 through cysteine residue(s) in the antibody polypeptide chains.

### 2. Treatment Regimens

In some embodiments, a ROR1 immunoconjugate described herein (e.g., ADC-A) is administered at a dose of 0.25 to 10 mg/kg, e.g., 0.25 to 4 mg/kg. For example, the immunoconjugate may be administered at a dose of 0.25 0.5, 0.75, 1, 1.25, 1.5, 1.75, 2, 2.25, 2.5, 2.75, 3, 3.25, 3.5, 3.75, 4, 4.25, 4.5, 4.75, 5, 5.5, 6, 6.5, 7, 8, 9, or 10 mg/kg, or any combination thereof for multiple doses. In certain embodiments, the immunoconjugate is administered at a dose of 0.50, 0.75, 1.00, 1.25, 1.50, 1.75, 2.00, 2.25, 2.50, 2.75, or 3.00 mg/kg.

In some embodiments, the immunoconjugate is administered in repeated cycles of 1, 2, 3, 4, 5, 6, 7, 8, 9, 10, 11, 12, 13, 14, 15, or 16 weeks. In certain embodiments, the immunoconjugate is administered in three-week cycles. In certain embodiments, the immunoconjugate is administered in four-week cycles. The treatment regimen may comprise 1, 2, 3, 4, 5, 6, 7, 8, 9, 10, 11, 12, 13, 14, 15, 16, 17, 18, 19, 20, or more cycles of administration (e.g., 3 or more cycles, or 4 or more cycles). In certain embodiments, the immunoconjugate is administered on one, two, three, four, five, six, or seven days of the cycle. The days of administration may be consecutive or may have one, two, three, four, five, or six days, one week, two weeks, three weeks, or four weeks, or any combination thereof, between them. In particular embodiments, the immunoconjugate is administered on Day 1 only of each cycle (e.g., a three-week cycle). In particular embodiments, the immunoconjugate is administered on Days 1 and 8 of each cycle (e.g., a three-week cycle). In particular embodiments, the immunoconjugate is administered on Days 1, 8, and 15 of each cycle (e.g., a four-week cycle).

The immunoconjugate may be administered initially according to a dosage regimen described herein and subsequently according to a different dosage regimen described herein (e.g., to increase or decrease the frequency of administration). In some embodiments, the immunoconjugate is administered weekly during the first 1, 2, 3, 4, 5, 6, 7, 8, 9, 10, 11, or 12 weeks, then every 3 weeks thereafter. In certain embodiments, the immunoconjugate is administered weekly during the first 2, 3, 4, 5, or 6 weeks, and then every 3 weeks. In certain embodiments, the immunoconjugate is administered weekly during the first 1, 2, 3, 4, 5, or 6 weeks, and then every 4 weeks. The immunoconjugate may be administered, e.g.:
weekly for the first three weeks and then every three weeks;
weekly for the first four weeks and then every three weeks;
weekly for the first six weeks and then every three weeks;
weekly for the first eight weeks and then every three weeks;
every three weeks for the first three weeks and then weekly;
every three weeks for the first six weeks and then weekly; or
every three weeks for the first nine weeks and then weekly.

In some embodiments, a dosage regimen described herein achieves an immunoconjugate plasma Cₘₐₓ of at least 10, 15, 20, 25, 30, 35, 40, 45, 50, 55, or 60 μg/mL in the patient. In some embodiments, a dosage regimen described herein achieves an immunoconjugate plasma area under the concentration-time curve (AUC) of at least 500, 750, 1000, 1250, 1500, 1750, 2000, 2250, 2500, 2750, 3000, 3250, or 3500 hour·μg/mL in the patient.

In some embodiments, a dosage regimen described herein maintains immunoconjugate occupancy of the ROR1 receptor of at least 30%, 40%, 50%, 60%, 70%, 75%, 80%, 85%, 90%, or 95% in the patient. In some embodiments, a dosage regimen described herein maintains at least 50% immunoconjugate occupancy of the ROR1 receptor for at least 20, 30, 40, 50, 60, 70, 80, or 90% of the time. In some embodiments, a dosage regimen described herein maintains at least 75% immunoconjugate occupancy of the ROR1 receptor for at least 20, 30, 40, 50, 60, 70, 80, or 90% of the time. In some embodiments, a dosage regimen described herein maintains at least 90% immunoconjugate occupancy of the ROR1 receptor for at least 0.5, 1, 1.5, 2, 2.5, 3, 3.5, 4, 4.5, or 5% of the time.

The immunoconjugate may be administered via parenteral administration. As used herein, “parenteral administration” of an immunoconjugate includes any route of administration characterized by physical breaching of a tissue of a subject and administration of the immunoconjugate through the breach in the tissue, thus generally resulting in the direct administration into the blood stream, into muscle, or into an internal organ. Parenteral administration thus includes, but is not limited to, administration of an immunoconjugate by injection of the immunoconjugate, by application of the immunoconjugate through a surgical incision, by application of the immunoconjugate through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intratumoral, and intrasynovial injection or infusions; and kidney dialytic infusion techniques. Regional perfusion is also contemplated. In some embodiments, the infusion may be administered by one route (e.g., intravenously) for initial doses and then be administered by another route for subsequent doses.

In certain embodiments, the immunoconjugate is administered by intravenous (IV) infusion. The IV infusion may take place over a period of about 0.1 to about 4 hours (e.g., about 5, 10, 15, 20, 25, 30, 35, 40, 45, 50, 55, 60, 65, 70, 75, 80, 85, 90, 120, or 180). In particular embodiments, the infusion time is 30 minutes. Infusion times may be extended as necessary to accommodate individual patient tolerance of treatment. Where the immunoconjugate is administered in more than one dose, in some embodiments, the infusion time for the first dose is longer than the infusion time for subsequent doses, or alternatively, the infusion time for the first dose is shorter than the infusion time for subsequent doses.

In some embodiments, the immunoconjugate is administered as a monotherapy.

It is understood that the treatment regimens of the invention may be methods of treatment as described herein, an immunoconjugate as described herein for use in a treatment regimen described herein, or use of an immunoconjugate as described herein for the manufacture of a medicament for use in a treatment regimen described herein.

### 3. Patient Selection

The treatment regimens of the invention may be used to treat a patient with cancer. In some embodiments, a treatment regimen of the invention includes the step of selecting a patient with a cancer described herein. In certain embodiments, the patient may have been treated previously for said cancer, and/or has a cancer that is relapsed or is refractory to one or more (or all) existing treatments for said cancer.

“Treat”, “treating” and “treatment” refer to a method of alleviating or abrogating a biological disorder and/or at least one of its attendant symptoms. As used herein, to “alleviate” a disease, disorder or condition means reducing the severity and/or occurrence frequency of the symptoms of the disease, disorder, or condition. Further, references herein to “treatment” include references to curative, palliative and prophylactic treatment. Treatment of cancer encompasses inhibiting cancer growth (including causing partial or complete cancer regression), inhibiting cancer progression or metastasis, preventing cancer recurrence or residual disease, and/or prolonging the patient's survival.

In some embodiments, the patient to be treated with a treatment regimen of the invention has a ROR1-expressing cancer. The ROR1-expressing cancer can be determined by any suitable method of determining gene or protein expression, for example, by histology, flow cytometry, radiopharmaceutical methods, RT-PCR, or RNA-Seq. The cancer cells used for the determination may be obtained through tumor biopsy or through collection of circulating tumor cells. In certain embodiments, if an antibody-based assay such as flow cytometry or immunohistochemistry is used, ROR1-expressing cancers are any cancers with cells that show anti-ROR1 antibody reactivity greater than that of an isotype control antibody. In certain embodiments, if an RNA-based assay is used, ROR1-expressing cancers are those that show an elevated level of ROR1 RNA compared to a negative control cell or cancer that does not express ROR1.

In certain embodiments, the patient has a hematological malignancy, such as a lymphoid malignancy. In certain embodiments, the patient has a solid tumor. The patient may have a cancer selected from, e.g., lymphoma, non-Hodgkin lymphoma, chronic lymphocytic leukemia (CLL), small lymphocytic lymphoma (SLL), marginal zone lymphoma (MZL), marginal cell B-cell lymphoma, Burkitt lymphoma (BL), mantle cell lymphoma (MCL), follicular lymphoma (FL), diffuse large B-cell lymphoma (DLBCL), a non-Hodgkin lymphoma that has undergone Richter's transformation, T cell leukemia, T cell lymphoma (e.g., T cell non-Hodgkin lymphoma), lymphoplasmacytic lymphoma (LPL), Waldenström macroglobulinemia (WM), acute myeloid leukemia (AML), acute lymphocytic leukemia (ALL), hairy cell leukemia (HCL), myeloma, multiple myeloma (MM), sarcoma (e.g., osteosarcoma, Ewing sarcoma, rhabdomyosarcoma, soft-tissue sarcoma, or uterine sarcoma), brain cancer, glioblastoma, astrocytoma, medulloblastoma, craniopharyngioma, ependymoma, neuroblastoma, head and neck cancer, nasopharyngeal cancer, thyroid cancer, breast cancer (e.g., ER/PR-positive breast cancer, HER2-positive breast cancer, or triple-negative breast cancer), lung cancer (e.g., non-small cell lung cancer or small cell lung cancer), malignant mesothelioma, bile duct/gall bladder cancer (e.g., cholangiocarcinoma), colon cancer, colorectal cancer, esophageal cancer, stomach cancer, gastric cancer, gastrointestinal stromal tumors (GIST), liver (hepatocellular) cancer, pancreatic cancer, renal cell carcinoma, bladder cancer, prostate cancer, cervical cancer, endometrial cancer, ovarian cancer, testicular cancer, epithelial squamous cell cancer, melanoma, adrenocortical carcinoma, gastrointestinal carcinoid tumors, islet cell tumors, pancreatic neuroendocrine tumors, neuroendocrine carcinoma of the skin (Merkel cell carcinoma), and pheochromocytoma. In certain embodiments, the patient has a cancer that is refractory to other therapeutics (e.g., triple negative breast cancer).

In particular embodiments, the patient has a cancer selected from CLL/SLL, MCL, FL, MZL, DLBCL, RTL, BL, LPL/WM, T cell NHL, ALL, and AML. In certain embodiments, the patient may have been treated previously for said cancer, and/or has a cancer that is relapsed or is refractory to one or more (e.g., all) existing treatments for said cancer.

In some embodiments, the patient is resistant to or has relapsed on treatment with ibrutinib, acalabrutinib, autologous hematopoietic stem cell transplantation, bendamustine, bortezomib, brentuximab vedotin, carmustine, chimeric antigen receptor T (CAR-T) cells, cisplatin, copanlisib, cyclophosphamide, cytarabine, daratumumab, dexamethasone, doxorubicin, etoposide, gemcitabine, idelalisib, lenalidomide, melphalan, methotrexate, methylprednisolone, mosunetuzumab, obinutuzumab, ofatumumab, oxaliplatin, pinatuzumab, polatuzumab, rituximab, prednisone, radiotherapy, venetoclax, vincristine, or any combination thereof (e.g., any combination of prior treatment agents found in the Examples).

In some embodiments, the treatment regimen is administered to a human patient, e.g., an adult patient (≥18 years of age), an adolescent patient (≥12 to 17 years of age), or a pediatric patient (<18 years of age) with adequate performance status and organ function who (i) has a histologically confirmed advanced hematological cancer or solid tumor; and/or (ii) has a malignancy that is unlikely to be responsive to established therapies known to provide clinical benefit, or has developed an intolerance to established therapies known to provide clinical benefit. In certain embodiments, the patient meets both criteria.

### 4. Treatment Outcomes

In some embodiments, treatment with the immunoconjugate results in one or more of the following:
induces partial or complete tumor regression, which may in some cases be sustained beyond the final dose of treatment;
delays tumor progression (e.g., by inhibiting tumor growth);
prevents cancer recurrence or residual disease;
decreases the size of nodal or extranodal tumor masses (that can be painful, disfiguring, or compressive);
decreases malignant cell numbers in bone marrow and peripheral blood;
decreases malignant splenomegaly or hepatomegaly;
improves cancer-related anemia, neutropenia, or thrombocytopenia (that can place patients at risk of fatigue, infection, or bleeding, respectively);
ameliorates cutaneous manifestation (that can be painful, intensely pruritic, or disfiguring);
decrease the likelihood of hyperviscosity syndrome in patients with LPL/WM;
ameliorates disabling constitutional symptoms; and
prolongs survival.
   Treatment may result in any combination of the above outcomes.

In some embodiments, a treatment regimen of the invention reduces tumor dimensions in a patient with a decrease of at least 10%, 20%, 30%, 40%, 50%, 60%, 70%, 80%, or 90% in the sum of the products of the perpendicular diameters (SPD). In some embodiments, a treatment regimen of the invention reduces tumor dimensions in a patient with a decrease of at least 10%, 20%, 30%, 40%, 50%, 60%, 70%, 80%, or 90% in the sum of the longest diameters of target lesions. In some embodiments, a treatment regimen of the invention completely eradicates the tumor.

In some embodiments, a treatment regimen of the invention (e.g., to treat CLL and/or SLL) results in one or more (e.g., any one, two, three, four, five, six, or seven) of the following.
a) no evidence of new disease;
b) absolute lymphocyte count (ALC) in peripheral blood of <4×10⁹/L;
c) regression of all index nodal masses to ≤15 mm in the longest diameter (LD);
d) normal spleen size of ≤120 mm by imaging in its longest vertical dimension (LVD) and normal liver size of ≤180 mm by imaging in its LVD;
e) regression to normal (e.g., less than ≤15 mm) of all nodal non-index disease and disappearance of all detectable non-nodal, non-index disease;
f) morphologically negative bone marrow defined as <30% of nucleated cells being lymphoid cells and no lymphoid nodules in a bone marrow sample that is normocellular for age; and
g) peripheral blood meeting all of the following criteria:
   i) absolute neutrophil count (ANC)>1.5×10⁹/L without exogenous growth factors ≤2 weeks before the relevant blood count assessment;
   ii) platelet count >100×10⁹/L without exogenous growth factors or platelet transfusions ≤2 weeks before the relevant blood count assessment; and
   iii) hemoglobin >110 g/L (11.0 g/dL) without exogenous growth factors or red blood cell transfusions ≤2 weeks before the relevant blood count assessment.
      In certain embodiments, the treatment regimen results in all of a)-g) (“complete response”), and may further result in flow cytometry of bone marrow aspirate showing malignant cells of ≤1×10⁻⁴ (“complete response without measurable residual disease”). In some embodiments, a treatment regimen of the invention results in one or more of the following (“complete response with incomplete count recovery”):
      a) a)-f), g)(ii), and g)(iii), and absolute neutrophil count (ANC)≤1.5/10⁹/L or requires exogenous growth factors ≤2 weeks before the relevant blood count assessment to maintain an ANC≥1.5×10⁹/L;
      b) a)-f), g)(i), and g)(iii), and platelet count ≤100×10⁹/L or requires exogenous growth factors or platelet transfusions ≤2 weeks before the relevant blood count assessment to maintain a platelet count ≥100×10⁹/L; and
      c) a)-f), g)(i), and g)(ii), and hemoglobin ≤110 g/L (11.0 g/dL) or requires exogenous growth factors or red blood cell transfusions ≤2 weeks before the relevant blood count assessment to maintain a hemoglobin ≥110 g/L (11.0 g/dL).

In some embodiments, a treatment regimen of the invention (e.g., to treat CLL/SLL) results in one or more (e.g., any one, two, three, or four) of the following:
a) no evidence of new disease;
b) a change in disease status meeting two or more of the following criteria, with the exception that if only lymphadenopathy is present at screening, only lymphadenopathy must improve to the extent specified below:
   i) decrease in peripheral blood absolute lymphocyte count (ALC) by 50% from screening;
   ii) a decrease by ≥50% from the screening in the sum of the products of the diameters (SPD) of the index nodal lesions;
   iii) in a subject with enlargement of the spleen at screening, a ≥50% decrease from screening (minimum decrease of 20 mm) in the enlargement of the spleen in its longest vertical dimension (LVD) or to ≤120 mm by imaging;
   iv) in a subject with enlargement of the liver at screening, a 50% decrease from screening (minimum decrease of 20 mm) in the enlargement of the liver in its LVD or to ≤180 mm by imaging; and
   v) a decrease by ≥50% from screening in the CLL/SLL bone marrow infiltrate or in B-lymphoid nodules;
c) no index, splenic, liver, or non-index disease with worsening that meets the criteria for definitive progressive disease (PD); and
d) peripheral blood meeting one or more of the following criteria:
   i) absolute neutrophil count (ANC)≥1.5×10⁹/L or ≥50% increase over screening without exogenous growth factors ≤2 weeks before the relevant blood count assessment;
   ii) platelet count >100×10⁹/L or ≥50% increase over screening without exogenous growth factors or platelet transfusions ≤2 weeks before the relevant blood count assessment; and
   iii) hemoglobin >110 g/L (11.0 g/dL) or ≥50% increase over screening without exogenous growth factors or red blood cell transfusions ≤2 weeks before the relevant blood count assessment.
      In certain embodiments, the treatment regimen results in all of a)-d) (“partial response). In certain embodiments, the treatment regimen results in all of a), b)ii)-b)v), c), and d) (“partial response with lymphocytosis”).

In some embodiments, a treatment regimen of the invention (e.g., to treat CLL/SLL) results in one or both of the following:
a) no evidence of new disease; and
b) no evidence of tumor growth.
   In certain embodiments, the treatment regimen results in both a) and b) (“stable disease”).

In some embodiments, a treatment regimen of the invention (e.g., to treat CLL/SLL) does not result in any of the following (which are signs of “progressive disease”):
a) evidence of any new disease, as determined by one or more of:
   i) a new node that measures >15 mm in any diameter;
   ii) a spleen LVD of >140 mm by imaging in subjects with a normal spleen LVD of ≤120 mm by imaging at nadir;
   iii) a liver LVD of >200 mm by imaging in subjects with a normal liver LVD of ≤180 mm by imaging at nadir;
   iv) histologically confirmed new effusions, ascites, or other organ abnormalities related to CLL/SLL;
   v) a new extranodal lesion >10 mm;
   vi) new fluorodeoxyglucose (FDG)-avid foci consistent with lymphoma rather than another etiology (e.g., infection or inflammation); and
   vii) new or recurrent bone marrow involvement with lymphoma by PET or by bone marrow biopsy if prior PET or bone marrow biopsy was negative for lymphoma;
b) evidence of worsening of index lesions, spleen or liver, or non-index disease, as determined by one or more of:
   i) increase from the nadir by ≥50% from the nadir in the sum of the products of the diameters (SPD) of index lesions;
   ii) increase from the nadir by ≥50% in the longest diameter (LD) of an individual node or extranodal mass that now has an LD of >15 mm and an LPD of >10 mm;
   iii) an increase in splenic enlargement by ≥50% (minimum increase of 20 mm) from nadir in subjects with splenomegaly at screening or at the splenic longest vertical dimension (LVD) nadir;
   iv) an increase in liver enlargement by ≥50% (minimum increase of 20 mm) from nadir in subjects with hepatomegaly at screening or at the hepatic LVD nadir;
   v) unequivocal increase in the size of effusions, ascites, or other organ abnormalities related to CLL/SLL; and
   vi) transformation to a more aggressive histology as established by lymph node biopsy;
   vii) decrease in platelet count or hemoglobin that is attributable to CLL/SLL, is not attributable to an autoimmune phenomenon, and is confirmed by bone marrow biopsy showing an infiltrate of clonal CLL/SLL cells, wherein
      A) the current platelet count is ≤100×10⁹/L and there has been a decrease by ≥50% from baseline; and
      B) the current hemoglobin is ≤110 g/L (11.0 g/dL) and there has been a decrease by >20 g/L (2 g/dL) from baseline.
         In certain embodiments, the treatment does not result in any of the above outcomes.

In some embodiments, a treatment regimen of the invention (e.g., to treat lymphoma) results in one or more (e.g., any one, two, three, four, five, six, seven, or eight) of the following.
a) no evidence of new disease;
b) regression of all index nodal lesions to ≤15 mm in the LDi;
c) regression to ≤15 mm of all nodal non-index disease;
d) disappearance of all detectable extranodal index and non-index disease;
e) normal spleen size of ≤130 mm by imaging in its longest vertical dimension (LVD);
f) If PET performed, no evidence of residual disease—i.e., score of 1 (no uptake above background), 2 (uptake ≤mediastinum), or 3 (uptake >mediastinum but ≤liver) on the Deauville 5-point scale;
g) negative for bone marrow involvement by PET for a PET-avid tumor or by morphological assessment of a unilateral core biopsy; if the bone marrow biopsy is indeterminate by morphology, it should be negative by immunohistochemistry; and
h) absence of serum M-protein by SIFE (in subjects with LPL/WM).
   In certain embodiments, the treatment regimen results in all of a)-g) (“complete response”) or a)-h) (“complete response” in subjects with LPL/WM), and may further result in flow cytometry of bone marrow aspirate showing malignant cells of ≤1×10⁻⁴ (“complete response without measurable residual disease”). In certain embodiments, the treatment regimen results in all of a)-h) as well as a ≥90% decrease from baseline in serum M-protein concentration.

In some embodiments, a treatment regimen of the invention (e.g., to treat lymphoma) results in one or more (e.g., any one, two, three, or four) of the following:
a) no evidence of new disease
b) a 50% decrease from screening in the SPD of the index nodal and extranodal lesions;
c) no increase from the nadir in the size of non-index disease;
d) in a subject with enlargement of the spleen at screening, a ≥50% decrease from screening (minimum decrease of 20 mm) in the enlargement of the spleen in its longest vertical dimension (LVD) or to ≤130 mm by imaging;
e) if PET is performed:
   i) typically FDG-avid lymphoma: if no screening PET scan was performed or if the PET scan was positive before therapy, the on-treatment PET is positive in ≥1 previously involved site—i.e., score of 4 (uptake moderately >liver) or score of 5 (uptake markedly >liver) on the Deauville 5-point scale but with reduced uptake compared with screening. If a screening PET was performed and was negative, there is no new PET evidence of disease. Reduced uptake is defined as a ≥25% decrease in the % ΔSUVmax;
   ii) variably FDG-avid lymphoma/FDG-avidity unknown: if no pretreatment PET scan was performed or if the pretreatment PET scan was negative for lymphoma, CT criteria should be used in assessing the tumor during treatment. If the PET scan was positive before therapy, the on-treatment PET is positive in ≥1 previously involved site;
f) persistence of bone marrow involvement in a subject who meets radiographic criteria for complete response (CR); and
g) a ≥50% but <90% decrease from baseline in serum M-protein concentration (in subjects with LPL/WM).
   In certain embodiments, the treatment regimen results in all of a)-f) (“partial response”) or a)-g) (“partial response” in subjects with LPL/WM). In certain embodiments, the treatment regimen results in all of a)-f) as well as a ≥25% but <50% decrease from baseline in serum M-protein concentration (“minor response” in LPL/WM).

In some embodiments, a treatment regimen of the invention (e.g., to treat lymphoma) results in one or more of the following:

a) no evidence of new disease;
b) no evidence of tumor growth; and
c) a <25% decrease and <25% increase from baseline in serum M-protein concentration (in subjects with LPL/WM).
In certain embodiments, a treatment regimen of the invention results in both a) and b) (“stable disease”) or a)-c) (“stable disease” in subjects with LPL/WM). In certain embodiments, a treatment regimen of the invention results in both a) and b) as well as a ≥25% but <50% decrease from baseline in serum M-protein concentration (“minor response” in LPL/WM).

In some embodiments, a treatment regimen of the invention does not result in one or more of the following (which are signs of “progressive disease”):
a) evidence of any new disease, as determined by one or more of:
   i) a new node that measures >15 mm in any diameter;
   ii) reappearance of an extranodal lesion that had resolved (ie, had previously been assigned a product of the perpendicular diameters (PPD) of 0 mm²);
   iii) a new extranodal lesion >10 mm;
   iv) new non-index disease (eg, effusions, ascites, or other organ abnormalities) of any size unequivocally attributable to lymphoma (e.g., as confirmed by PET, biopsy, cytology, or other non-radiologic assays);
   v) new FDG-avid foci consistent with lymphoma rather than another etiology (e.g., infection or inflammation; if there is uncertainty regarding the etiology of new lesions, biopsy or interval scan may be considered); and
   vi) new or recurrent bone marrow involvement with lymphoma by PET or by bone marrow biopsy if prior PET or bone marrow biopsy performed as part of the study was negative for lymphoma;
b) evidence of worsening of nodal or extranodal index lesions, as determined by one or more of:
   i) increase from the nadir by ≥50% from the nadir in the sum of the products of the diameters (SPD) of index lesions;
   ii) evidence of worsening of individual index lymph nodes or nodal masses with an increase from the nadir by ≥50% in the PPD for any individual node if the node now has an LD of >15 mm, an increase by ≥50% from the nadir PPD, and an increase in LD or SD from the nadir by ≥5 mm for lesions measuring ≤20 mm (in LD or shortest dimension (SDi)) or ≥10 mm for lesions measuring >20 mm (in LD or SDi)
   iii) unequivocal increase in the size of non-index disease; and
   iv) an increase in splenic enlargement by ≥50% (minimum increase of 20 mm) from nadir in a patient with a spleen LVD of >130 mm by imaging at nadir, or a spleen LVD of >150 mm by imaging in a patient with a spleen LVD of ≤130 mm by imaging at nadir;
   c) transformation to a more aggressive non-Hodgkin lymphoma (NHL) histology as established by lymph node biopsy;
   d) if PET is performed, there is a score of 4 (uptake moderately >liver) or score of 5 (uptake markedly >liver) on the Deauville 5-point scale with an increase in uptake compared with the nadir in conjunction with an anatomic increase in lesion size consistent with progressive disease (PD). Increased uptake is defined as a ≥50% increase in the % ΔSUVmax; and
   e) An increase from the nadir by ≥25% in serum M-protein concentration (in subjects with LPL/WM).
      In certain embodiments, the treatment does not result in any of a)-d), or does not result in any of the above outcomes.

In some embodiments, a treatment regimen of the invention (e.g., to treat acute lymphoid leukemia) results in one or more (e.g., any one, two, or three) of the following:
a) leukemia disease status meeting all of the following requirements:
   i) <5% bone marrow blasts (based on a bone marrow aspirate/biopsy sample with ≥200 nucleated cells and the presence of bone marrow spicules);
   ii) no blasts in the peripheral blood; and
   iii) no extramedullary disease (including lymphadenopathy, splenomegaly, skin/gum infiltration, testicular mass, and no central nervous system involvement (i.e., attainment of CNS-1 status (no blasts in the cerebrospinal fluid)));
b) peripheral blood meeting all of the following requirements:
   i) ANC≥1.0×10⁹/L;
   ii) platelet count ≥100×10⁹/L; and
c) any mediastinal enlargement shows complete resolution as documented radiographically.
   In certain embodiments, the treatment regimen results in all of a)-c) (“complete response”), and may further result in flow cytometry of a bone marrow aspirate showing malignant cells of ≤1×10⁻⁴ (“complete response without measurable residual disease”).

In some embodiments, a treatment regimen of the invention (e.g., to treat acute lymphoid leukemia) results in one or more (e.g., any one, two, or three) of the following:
a) leukemia disease status meeting all of the following requirements:
   i) <5% bone marrow blasts (based on a bone marrow aspirate/biopsy sample with ≥200 nucleated cells and the presence of bone marrow spicules);
   ii) no blasts in the peripheral blood; and
   iii) no extramedullary disease (including lymphadenopathy, splenomegaly, skin/gum infiltration, testicular mass, and no central nervous system involvement (i.e., attainment of CNS-1 status (no blasts in the cerebrospinal fluid)));
b) peripheral blood meeting all of the following requirements:
   i) ANC<1.0×10⁹/L;
   ii) platelet count <100×10⁹/L; and
c) any mediastinal enlargement has regressed by ≥75% in the sum of the products of the perpendicular diameters (SPD) as documented radiographically.

In certain embodiments, the treatment regimen results in all of a)-c) (“complete response with incomplete blood count recovery” and/or “complete response unconfirmed”).

In some embodiments, a treatment regimen of the invention (e.g., to treat acute lymphoid leukemia) results in one or more (e.g., any one, two, or three) of the following:
a) leukemia disease status meeting either of the following requirements:
   i) a ≥50% decrease in bone marrow blasts to 5% to 25% (based on a bone marrow aspirate/biopsy sample with ≥200 nucleated cells and the presence of bone marrow spicules);
   ii) no blasts in the peripheral blood; and
   iii) no new or worsening extramedullary disease (including lymphadenopathy, splenomegaly, skin/gum infiltration, testicular mass, and no central nervous system involvement (e.g., CNS-1 status (no blasts in the cerebrospinal fluid) has not transitioned to CNS-2 status (WBC<5×10⁹/L with presence of blasts in the cerebrospinal fluid) or to CNS-3 status (WBC≥5×10⁹/L with presence of blasts in the cerebrospinal fluid) or to development of facial nerve palsy, brain/eye involvement, or hypothalamic syndrome));
b) peripheral blood meeting all of the following requirements:
   i) ANC≥1.0×10⁹/L;
   ii) platelet count ≥100×10⁹/L; and
c) any mediastinal enlargement has regressed by ≥50% in the SPD as documented radiographically.

In certain embodiments, the treatment regimen results in all of a)-c) (“partial response”).

In some embodiments, a treatment regimen of the invention (e.g., to treat acute lymphoid leukemia) results in one or more (e.g., one, two, or three) of the following:

a) neither sufficient ALL improvement from baseline to qualify for PR nor sufficient evidence of ALL worsening to qualify for DRP;
b) no new or worsening extramedullary disease (including lymphadenopathy, splenomegaly, skin/gum infiltration, testicular mass, and no central nervous system involvement (e.g., CNS-1 status (no blasts in the cerebrospinal fluid) has not transitioned to CNS-2 status (WBC<5×10⁹/L with presence of blasts in the cerebrospinal fluid) or to CNS-3 status (WBC≥5×10⁹/L with presence of blasts in the cerebrospinal fluid) or to development of facial nerve palsy, brain/eye involvement, or hypothalamic syndrome));
c) no development of new mediastinal enlargement and no increase in the SPD of existing mediastinal enlargement by >25%.
In certain embodiments, a treatment regimen of the invention results in all of a)-c) (“stable disease”).

In some embodiments, a treatment regimen of the invention (e.g., to treat acute lymphoid leukemia) does not result in one or more (e.g., one, two, three, four, five, or six) of the following (which are signs of disease recurrence or progression, “DRP”):
a) reappearance of bone marrow blasts (to >5%) in a subject who had experienced a CR;
b) reappearance of blasts in the peripheral blood in a subject who had experienced a CR;
c) a 225% increase in bone marrow blasts to ≥20% (based on a bone marrow aspirate/biopsy sample with ≥200 nucleated cells and the presence of bone marrow spicules);
d) a 225% increase in blasts in the peripheral blood to >1×10⁹/L;
e) development of new or worsening existing extramedullary disease (involving lymphadenopathy, splenomegaly, skin/gum infiltration, testicular mass, or CNS involvement (e.g., CNS-1 status (no blasts in the cerebrospinal fluid) has transitioned to CNS-2 status (WBC<5×10⁹/L with presence of blasts in the cerebrospinal fluid) or to CNS-3 status (WBC≥5×10⁹/L with presence of blasts in the cerebrospinal fluid) or to development of facial nerve palsy, brain/eye involvement, or hypothalamic syndrome)); and
f) development of new mediastinal enlargement or increase in the SPD of existing mediastinal enlargement by ≥25%.

In certain embodiments, the treatment does not result in any of a)-f).

In some embodiments, a treatment regimen of the invention (e.g., to treat acute myeloid leukemia) results in one or both of the following:
a) leukemia disease status meeting all of the following requirements:
   i) <5% bone marrow blasts (based on a bone marrow aspirate/biopsy sample with ≥200 nucleated cells and the presence of bone marrow spicules);
   ii) no blasts in the peripheral blood;
   iii) no blasts with Auer rods; and
   iv) no extramedullary disease; and
b) peripheral blood meeting both of the following requirements:
   i) ANC 21.0×10⁹/L;
   ii) platelet count ≥100×10⁹/L.
      In certain embodiments, the treatment regimen results in both a) and b) (“complete response”), and may further result in flow cytometry of a bone marrow aspirate showing malignant cells of ≤1=10⁻⁴ (“complete response without measurable residual disease”).

In some embodiments, a treatment regimen of the invention (e.g., to treat acute myeloid leukemia) results in one or both of the following:
a) leukemia disease status meeting all of the following requirements:
   i) <5% bone marrow blasts (based on a bone marrow aspirate/biopsy sample with ≥200 nucleated cells and the presence of bone marrow spicules);
   ii) no blasts in the peripheral blood;
   iii) no blasts with Auer rods; and
   iv) no extramedullary disease; and
b) peripheral blood meeting only one of the following criteria:
   i) ANC≥1.0×10⁹/L; and
   ii) platelet count ≥100×10⁹/L.
      In certain embodiments, the treatment regimen results in both a) and b) (“complete response with incomplete blood count recovery”).

In some embodiments, a treatment regimen of the invention (e.g., to treat acute myeloid leukemia) results in one or both of the following:
a) leukemia disease status meeting all of the following requirements:
   i) <5% bone marrow blasts (based on a bone marrow aspirate/biopsy sample with ≥200 nucleated cells and the presence of bone marrow spicules);
   ii) no blasts in the peripheral blood;
   iii) no blasts with Auer rods; and
   iv) no extramedullary disease; and
b) peripheral blood meeting both of the following criteria:
   i) ANC≤1.0×10⁹/L; and
   ii) platelet count ≤100×10⁹/L.
      In certain embodiments, the treatment regimen results in both a) and b) (“morphologic leukemia-free state”).

In some embodiments, a treatment regimen of the invention (e.g., to treat acute myeloid leukemia) results in one or both of the following:
a) leukemia disease status meeting either of the following requirements:
   i) a ≥50% decrease in bone marrow blasts to 5% to 25% (based on a bone marrow aspirate/biopsy sample with ≥200 nucleated cells and the presence of bone marrow spicules);
   ii) ≤5% bone marrow blasts but with Auer rods present (based on a bone marrow aspirate/biopsy sample with ≥200 nucleated cells and the presence of bone marrow spicules);
   iii) no blasts in the peripheral blood; and
   iv) no new or worsening extramedullary disease; and
b) peripheral blood meeting both of the following criteria:
   i) ANC≥1.0×10⁹/L; and
   ii) platelet count ≥100×10⁹/L.
      In certain embodiments, the treatment regimen results in both a) and b) (“partial response”).

In some embodiments, a treatment regimen of the invention (e.g., to treat acute myeloid leukemia) results in one or both of the following:
a) neither sufficient AML improvement from baseline to qualify for CR_{MRD-}, CR, CRi, MLFS, or PR, nor sufficient evidence of AML worsening to qualify for DRP; and
b) no new or worsening extramedullary disease.
   In certain embodiments, a treatment regimen of the invention results in both a) and b) (“stable disease”).

In some embodiments, a treatment regimen of the invention (e.g., to treat acute myeloid leukemia) does not result in one or more of the following (which are signs of disease recurrence or progression, “DRP”):
a) reappearance of bone marrow blasts (to >5%) in a subject who had experienced a CR_{MRD-}, CR, CRi, or MLFS;
b) reappearance of blasts in the peripheral blood in a subject who had experienced a CR_{MRD-}, CR, CRi, MLFS, or PR;
c) an absolute 20% increase in bone marrow blasts to ≥25% (based on a bone marrow aspirate/biopsy sample with ≥200 nucleated cells and the presence of bone marrow spicules) in a subject who had experienced a PR;
d) an absolute 20% increase in peripheral blasts to >25% in a subject who had experienced a PR; and
e) development of new extramedullary disease or worsening of existing extramedullary disease.
   In certain embodiments, the treatment does not result in any of a)-e).

In some embodiments, a treatment regimen of the invention (e.g., to treat a solid tumor) results in one or both of the following:

a) disappearance of all target lesions and no new measurable lesions (e.g., lesions that can be accurately measured in ≥1 dimension (longest diameter to be recorded) as >10 mm with CT scan with minimum slice thickness of 5 mm) or >10 mm caliper measurement by clinical exam, or >20 mm by chest X-ray); and
b) reduction in the short axis to <10 mm for any pathological lymph nodes, whether target (e.g., ≥15 mm in short axis when assessed by CT scan (minimum slice thickness of 5 mm)) or nontarget (e.g., ≥10 mm but <15 mm).
In certain embodiments, the treatment regimen results in both a) and b) (“complete response” for target lesions).

In some embodiments, a treatment regimen of the invention (e.g., to treat a solid tumor) results in a ≥30% decrease in the sum of the diameters of target lesions taking as a reference the baseline sum of the diameters and including any new measurable lesions that may have appeared since baseline (“partial response” for target lesions).

In some embodiments, a treatment regimen of the invention (e.g., to treat a solid tumor) results in a ≥30% decrease in the sum of the diameters of target lesions taking as a reference the baseline sum of the diameters and including any new measurable lesions that may have appeared since baseline (“partial response” for target lesions).

In some embodiments, a treatment regimen of the invention (e.g., to treat a solid tumor) results in neither sufficient shrinkage to qualify for a partial response, taking as a reference the baseline sum of the diameters, nor sufficient increase to qualify for progressive disease, taking as a reference the smallest sum of the diameters during treatment (“stable disease” for target lesions).

In some embodiments, a treatment regimen of the invention (e.g., to treat a solid tumor) does not result in a ≥20% increase (and an absolute increase of ≥5 mm) in the sum of the diameters of the target lesions (including any new lesions), taking as a reference the smallest post-baseline sum (nadir tumor burden) or baseline sum if that is the smallest sum during treatment (“progressive disease” for target lesions).

In some embodiments, a treatment regimen of the invention (e.g., to treat a solid tumor) results in one or more of the following:

a) disappearance of all nontarget lesions;
b) normalization of an elevated tumor marker level; and
c) all lymph nodes nonpathologic in size (<10 mm in the short axis).
In certain embodiments, the treatment regimen results in all of a)-c) (“complete response” for nontarget lesions). In certain embodiments, the treatment regimen results in b) and c) but not a), or a) and c) but not b) (“non-complete response/non-progressive disease” for nontarget lesions).

In some embodiments, a treatment regimen of the invention (e.g., to treat a solid tumor) does not result in unequivocal progression of existing nontarget lesions (“progressive disease” for nontarget lesions”).

In some embodiments, a treatment regimen of the invention (e.g., to treat a solid tumor) results in an overall response of:
absence of target lesions and new measurable lesions; absence of nontarget lesions or tumor markers; and absence of new, nonmeasurable lesions (“complete response”);
absence of target lesions and new measurable lesions; and stable nontarget lesions or tumor markers (“partial response”);
absence of target lesions and new measurable lesions; and unequivocal progression in nontarget lesions or tumor markers (“partial response”);
decrease ≥30% in target lesions and new measurable lesions; and absent, stable, or unequivocal progression in nontarget lesions or tumor markers (“partial response”); or
decrease <30% to increase <20% in target lesions and new measurable lesions; and absent, stable, or unequivocal progression in nontarget lesions or tumor markers (“stable disease”).
   In some embodiments, a treatment regimen of the invention (e.g., to treat a solid tumor) does not result in an increase ≥20/% in target lesions and new measurable lesions; and absent, stable, or unequivocal progression in nontarget lesions or tumor markers (“progressive disease”).

In some embodiments, a treatment regimen of the invention results in one or more of the following outcomes, e.g., as defined above or in the Examples:
CLL/SLL: a CR, CRi, PR, or PR-L;
FL, MZL, MCL, DLBCL, or BL: a CR or PR;
LPL/WM: a CR, VGPR, PR, or MR;
ALL: a CR, CRi/CRu, or PR;
AML: a CR, CRi, or PR; or
solid tumor: a CR or PR.

In some embodiments, a treatment regimen of the invention does not result in one or more (e.g., any one, two, three, four, five, six, seven, eight, nine, or ten) of the following in the first cycle of treatment:
a) Grade ≥3 febrile neutropenia;
b) Grade ≥4 neutropenia;
c) Grade 3 thrombocytopenia with Grade ≥3 bleeding;
d) Grade ≥4 thrombocytopenia;
e) Grade 3 tumor lysis syndrome (TLS) despite adequate prophylaxis that does not resolve within 72 hours from onset;
f) Grade ≥4 TLS despite adequate prophylaxis;
g) Grade ≥3 vomiting despite recommended (or equivalent) antiemetic support,
h) Grade ≥3 nonhematological laboratory abnormalities that do not improve to Grade ≤1 or baseline within 72 hours;
i) Other Grade ≥3 nonhematological treatment-emergent adverse events (TEAEs) (with the exception of Grade 3 fatigue); and
j) Failure to recover to baseline by ≥21 days from the last dose of the immunoconjugate in the cycle due to a drug-related TEAE,
   wherein all grades are defined using the Common Terminology Criteria for Adverse Events (CTCAE), Version 5.0. In certain embodiments, a treatment regimen of the invention does not result in any of said outcomes.

In some embodiments, a treatment regimen of the invention does not result in one or more (e.g., any one, two, three, four, five, or six) of the following:
a) any complication that occurs as a result of a protocol-mandated procedure (e.g., venipuncture, ECG);
b) any preexisting condition that increases in severity or changes in nature during or as a consequence of drug administration (e.g., worsening manifestations of the underlying cancer, such as an increase in pain, tumor flare reaction, TLS, etc.);
c) any injury or accident;
d) any abnormality in physiological testing or a physical examination finding that requires clinical intervention or further investigation (beyond ordering a repeat or confirmatory test);
e) any laboratory (e.g., clinical chemistry, hematology, urinalysis) or investigational (e.g., ECG, X-ray) abnormality independent of the underlying medical condition that requires clinical intervention, results in further investigation (beyond ordering a repeat or confirmatory test), or leads to investigational medicinal product interruption or discontinuation unless it is associated with an already reported clinical event; and
f) a complication related to pregnancy or termination of a pregnancy.
   In certain embodiments, a treatment regimen of the invention does not result in any of said outcomes.

In some embodiments, a treatment regimen of the invention does not result in one or more (e.g., any one, two, three, four, five, or six) of the following:
a) death;
b) a life-threatening situation with an immediate risk of death;
c) in-patient hospitalization or prolongation of existing hospitalization;
d) persistent or significant disability/incapacity;
e) congenital anomaly/birth defect in the offspring of a subject who received the immunoconjugate; and
f) a medically significant event that may not be immediately life-threatening or result in death or hospitalization, but based upon appropriate medical and scientific judgment, may jeopardize the subject or may require medical or surgical intervention to prevent one of the outcomes listed above (e.g., allergic bronchospasm requiring intensive treatment in an emergency room or at home, new cancers or blood dyscrasias, convulsions that do not result in hospitalization, or development of drug dependency or drug abuse).
   In certain embodiments, a treatment regimen of the invention does not result in any of said outcomes.

In some embodiments, a treatment regimen of the invention does not result in one or more (e.g., one, two, or three) of the following:

a) Grade ≥3 infusion reactions;
b) tumor lysis syndrome (TLS) of any grade; and
c) Grade ≥3 peripheral neuropathy;
wherein all grades are defined using the Common Terminology Criteria for Adverse Events (CTCAE), Version 5.0.
In certain embodiments, a treatment regimen of the invention does not result in any of said outcomes.

In some embodiments, a treatment regimen of the invention does not result in abnormalities in one or more (e.g., any one, two, three, four, five, six, seven, eight, or nine) of the following: urine, serum, blood, systolic blood pressure, diastolic blood pressure, pulse, body temperature, blood oxygen saturation, and electrocardiography (ECG) readings. In certain embodiments, a treatment regimen of the invention does not result in abnormalities of any of the above.

In some embodiments, a treatment regimen of the invention does not result in detectable levels of circulating immunoconjugate-reactive antibodies in the patient's serum.

It is understood that the treatment regimens described herein may be methods of treatment as described herein, an immunoconjugate as described herein for use in a treatment regimen described herein, or use of an immunoconjugate as described herein for the manufacture of a medicament for a treatment regimen described herein.

### 5. Premedications or Concurrent Medications

### Tumor Lysis Syndrome Prophylaxis

In some embodiments, a patient to be treated with a treatment regimen of the invention is assessed for risk of tumor lysis syndrome (TLS) using the following criteria:

### CLL/SLL and NHL:

(i) Low-risk: Serum lactate dehydrogenase (LDH)≤upper limit of normal (ULN), all measurable lymph nodes <5 cm diameter, and ALC<25×10⁹/L.
(ii) Intermediate risk: Serum LDH>1 to ≤2×ULN, ≥1 measurable lymph node of ≥5 but <10 cm diameter, or ALC≥25×10⁹/L.
(iii) High risk: Serum LDH>2×ULN, ≥1 measurable lymph node of ≥10 cm diameter, or both ≥1 measurable lymph node with an LD of ≥5 but <10 cm diameter and ALC≥25×10⁹/L. ALL:
(i) Low risk: white blood count (WBC)<20×10⁹/L and serum LDH level <2×ULN
(ii) Intermediate risk: WBC≥20×10⁹/L to <100×10⁹/L and serum LDH level <2×ULN
(iii) High risk: WBC≥100×10⁹/L or serum LDH level ≥2×ULN AML:
(i) Low risk: Serum LDH<2×ULN, serum uric acid <5.5 mg/dL, and WBC<25×10⁹/L.
(ii) Intermediate risk: Serum LDH≥2×ULN, or serum uric acid 25.5 mg/dL and <7 mg/dL, or WBC≥25×10⁹/L to <100×10⁹/L.
(iii) High risk: Serum uric acid ≥7 mg/dL or WBC≥100×10⁹/L.

In some embodiments, if a patient is at intermediate or high risk of TLS, and/or if TLS is observed during treatment, the patient may receive allopurinol and/or febuxostat before or during treatment. A patient with hyperuricemia may additionally receive rasburicase. For example, the patient may receive said drug(s) according to a regimen described below:

(i) Intermediate Risk of TLS: The patient may receive allopurinol, 100 to 300 mg orally every 8 hours starting ≥24 to 48 hours before the start of drug therapy; of note, the maximum daily allopurinol dose is 800 mg, doses ≤300 mg need not be divided, and doses should be reduced by ≥50% in subjects with renal insufficiency. Alternative drugs (eg, febuxostat) may be substituted, with administration per product labelling. In addition, patients with hyperuricemia may receive rasburicase, 3 to 4.5 mg by IV infusion.

(ii) High Risk of TLS: The patient may receive allopurinol, 100 to 300 mg orally every 8 hours starting ≥24 to 48 hours before the start of drug therapy; of note, the maximum daily allopurinol dose is 800 mg, doses ≤300 mg need not be divided (but may be insufficient for high-risk subjects), and doses should be reduced by 50% in subjects with renal insufficiency. Alternative drugs (eg, febuxostat) may be substituted, with administration per product labelling. In addition, high risk patients may receive rasburicase, 3 to 4.5 mg by IV infusion, administered 3 to 4 hours prior to the first dose of drug.

In some embodiments, patients are monitored for TLS during Cycle 1, Day 1 (C1D1) through C1D3 with assessments of vital signs, AEs, and serum chemistry and hematology laboratory studies.

### Infusion Reaction Prophylaxis

In some embodiments, before or while receiving a treatment regimen of the invention, a patient may receive an antipyretic and/or an antihistamine to reduce the incidence and severity of infusion reactions. In certain embodiments, the antipyretic may be administered by the oral or IV route, and may be, e.g., acetaminophen (paracetamol), 650 to 1,000 mg or equivalent. In certain embodiments, the antihistamine may be administered by the oral or IV route, and may be, e.g., cetirizine, 10 mg or equivalent. In some embodiments, one or both drugs are given 30 to 60 minutes prior to each immunoconjugate infusion. A nonsteroidal antiinflammatory drug (NSAID) such as ibuprofen, 400 to 800 mg orally or equivalent, may be added or substituted for acetaminophen. A corticosteroid such as prednisolone, 100 mg or equivalent, may also be considered as a premedication.

### Antiemetic Prophylaxis

In some embodiments, before or while receiving a treatment regimen of the invention, a patient may receive an antiemetic to treat nausea and/or vomiting.

Neutropenia Management

In some embodiments, before or while receiving a treatment regimen of the invention, a patient may receive G-CSF (e.g., filgrastim, frastim-SND, PEG-filgrastim, or lenograstim) or GM-CSF (e.g., sargramostim) to prevent or mitigate drug-induced neutropenic complications and promote neutrophil recovery.

### Hyperviscosity Syndrome (HVS) Management

HVS is a clinical feature in 10% to 30% of patients with LPL/WM due to the presence of high levels of circulating M-protein. Immediate therapy of symptomatic HVS is typically plasmapheresis. In some embodiments, before or while receiving a treatment regimen of the invention, a patient may receive plasmapheresis to prevent or mitigate HVS.

### 6. Articles of Manufacture and Kits

The present invention also provides articles of manufacture, e.g., kits, comprising one or more containers (e.g., single-use or multi-use containers) containing a pharmaceutical composition of an immunoconjugate described herein at a dose described herein, optionally an additional biologically active molecule (e.g., another therapeutic agent), and instructions for use according to a treatment regimen described herein. The immunoconjugate and additional biologically active molecule can be packaged together or separately in suitable packing such as a vial or ampule made from non-reactive glass or plastic. In some embodiments, the vial or ampule holds a liquid containing the immunoconjugate or a lyophilized powder comprising the immunoconjugate; the liquid or lyophilized powder may optionally include the additional therapeutic agent or biologically active molecule. In certain embodiments, the vial or ampule holds a concentrated stock (e.g., 2×, 5×, 10× or more) of the immunoconjugate and optionally the biologically active molecule. In particular embodiments, a pharmaceutical composition of an immunoconjugate described herein (e.g., ADC-A) is packaged in a single-use glass vial containing 50 mg, 100 mg, 150 mg, 200 mg, 250 mg, or 300 mg of the immunoconjugate (e.g., appropriate for use at a dose described herein, such as 0.50, 0.75, 1.00, 1.25, 1.50, 1.75, 2.00, 2.25, 2.50, 2.75, or 3.00 mg/kg). In certain embodiments, the articles of manufacture such as kits include a medical device for administering the immunoconjugate and/or biologically active molecule (e.g., a syringe and a needle); and/or an appropriate diluent (e.g., sterile water and normal saline). The present invention also includes methods for manufacturing said articles.

Unless the context requires otherwise, throughout the specification and claims, the word “comprise” and variations thereof, such as, “comprises” and “comprising,” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. As used herein the term “about” refers to a numerical range that is 10%, 5%, or 1% plus or minus from a stated numerical value within the context of the particular usage. Further, headings provided herein are for convenience only and do not interpret the scope or meaning of the claimed embodiments.

All publications and patents mentioned herein are incorporated herein by reference in their entirety for the purpose of describing and disclosing, for example, the constructs and methodologies that are described in the publications, which might be used in connection with the presently described inventions. The publications discussed herein are provided solely for their disclosure prior to the filing date of the present application. Nothing herein is to be construed as an admission that the inventors of the subject invention are not entitled to antedate such disclosure by virtue of prior invention or for any other reason.

Unless defined otherwise, all technical and scientific terms used herein have the same meaning as commonly understood to one of ordinary skill in the art to which the inventions described herein belong. Any methods, devices, and materials similar or equivalent to those described herein can be used in the practice or testing of the inventions described herein.

### EXAMPLES

The following examples illustrate representative embodiments of the present invention and are not meant to be limiting in any way.

### Example 1: Synthesis of ADC-A

Conjugation of Ab1 with MC-VC-PAB-MMAE (ADC-A) was performed at multiple scales (from 2 mg to 200 g) with similar results. At a large scale, approximately 200 g of Ab1 (approximately 40 mg/mL in 50 mM sodium citrate, 10 mg/mL trehalose, 0.05 mM EDTA, 0.02% polysorbate 80, pH 5.2) was treated with 1.90 equivalents (eq) of tris(2-carboxyethyl)phosphine (TCEP, 5 mM) and held at 20-24° C. for 330 minutes. Next, 6.5 eq MC-VC-PAB-MMAE in N,N-dimethylacetamide (DMA) was added and the mixture was held at 22-23° C. for an additional 60 minutes. The buffer was exchanged with 10 mM sodium acetate, pH 4.8 by ultrafiltration/diafiltration (UF/DF) using 30 kD UF membrane cassettes. The number of MMAE drug molecules linked per antibody molecule (DAR) was determined using HIC-HPLC. Data from HIC-HPLC, SEC-HPLC, RP-HPLC, and UV/Vis are summarized in Table 1 below. Consistent results were obtained at all scales, with DAR ranging from 3.89 to 5.09 on average, depending on the methodology used.

TABLE 1
ADC-A Drug-Antibody Ratio (DAR)

| | Aggregate | Recovery | | DAR | | | |
|---|---|---|---|---|---|---|---|
| Scale | (%) | (%) | D0* (%) | UV | SEC | HIC | RP |
| 2 mg | 3.47 | 85.6 | 6.06 | 4.33 | 4.81 | 3.95 | 3.89 |
| 30 mg | 3.50 | 86.1 | 4.75 | 4.44 | 5.09 | 4.21 | 4.09 |
| 350 mg | 3.51 | 90.2 | 5.12 | 4.40 | 4.96 | 4.12 | 3.99 |
| 200 g | 2.1 | 95.4 | 4.9 | | | 4.0 | |

*D0: unconjugated antibody.

### Example 2: Dose-Escalation Study for Anti-ROR1-MMAE Immunoconjugates

The following describes a protocol for evaluating the safety, pharmacokinetics, pharmacodynamics, immunogenicity, and efficacy of a ROR1 immunoconjugate (ADC-A) across a range of dose levels when administered to subjects with previously treated relapsed or refractory CLL/SLL, MCL, FL, MZL, DLBCL, RTL, BL, LPL/WM, T cell NHL, ALL, or AML. ADC-A is administered intravenously (IV) in repeated 3-week cycles with a drug infusion on Day 1 of each cycle (Q1/3W [Schedule 1]); in repeated 3-week cycles with drug infusions on Days 1 and 8 of each cycle (Q2/3W [Schedule 2]); or in repeated 4-week cycles with drug infusions on Days 1, 8, and 15 of each cycle (Q3/4W [Schedule 3]) over a planned infusion time of ˜30 minutes. Infusion times may be extended as necessary to accommodate individual subject tolerance of treatment.

TABLE 2
ADC-A Dosing Schedules

| | | Days of Dosing | |
|---|---|---|---|
| Schedule | Duration of Cycle | During Cycle | Designation |
| 1 | 3 weeks | Day 1 | Q1/3W |
| 2 | 3 weeks | Days 1 and 8 | Q2/3W |
| 3 | 4 weeks | Days 1, 8, and 15 | Q3/4W |

### Dose Levels

The initial cohort of subjects will be prescribed ADC-A at 0.50 mg/kg Q1/3W. Thereafter, cohorts of subjects will be sequentially enrolled at progressively higher starting dose levels of ADC-A to be administered Q1/3W, Q2/3W, or Q3/4W (Tables 2 and 3). An initial dose of 0.25 mg/kg may be administered in the Q1/3W schedule to permit a dose decrement if a subject experiences a TEAE requiring dose modifications to a level below the starting level.

TABLE 3
ADC-A Dose Levels

| Q1/3W | | Q2/3W | | Q3/4W |
|---|---|---|---|---|
| 0.50 | | 0.75* | | 0.75* |
| 1.00 | Starting Level | 1.00 | Starting Level | 1.00 |
| 1.50 | | 1.25 | | 1.25 |
| 2.25 | | 1.50 | | 1.50 |
| 2.50 | | 1.75 | | 1.75 |
| 2.75^{§} | | 2.00 | | 2.00 |
| 3.00^{§} | | 2.25 | | 2.25 |

*The starting dose level is 1.00 mg/kg. The 0.75 mg/kg dose is provided to permit a dose decrement if a subject experiences a treatment-emergent adverse event (TEAE) requiring dose modifications to a level below 1.00 mg/kg.
^{§}May be administered if subjects tolerate ADC-A therapy at the prior dose level.

An accelerated dose escalation in a single subject at the initial dose level using the Q1/3W schedule is planned. Thereafter, cohorts of 3 to 6 subjects will be sequentially enrolled evaluating each schedule of administration at progressively higher dose levels of ADC-A using a standard 3+3 dose-escalation design. Based on the pattern of dose-limiting toxicities (DTLs) observed in Cycle 1, escalation will proceed to define a maximum-tolerated dose (MTD) and a recommended dosing regimen (RDR) for each schedule of administration that may be at the MTD or a lower dose within the tolerable dose range. The MTD is the highest tested dose level at which ≥6 subjects have been treated and which is associated with a Cycle 1 dose-limiting toxicity (DLT) in ≤17% of the subjects. The RDR may be the MTD or may be a lower dose within the tolerable dose range. Selection of each RDR will be based on consideration of short- and long-term safety information together with available pharmacokinetic, pharmacodynamic, and efficacy data, and may be defined in the context of the level of supportive care (eg, antiemetic or hematopoietic prophylaxis) provided to subjects to achieve the RDR. Once each RDR has been established, further development will be considered in specific hematological cancers and/or solid tumors.

It is expected that ADC-A administered to the patients in accordance with the dosing regimens provided will achieve overall response (OR), defined as achievement of the following outcomes by disease type:
CLL/SLL: Complete response (CR), complete response with incomplete blood count recovery (CRi), partial response (PR), or partial response with lymphocytosis (PR-L);
NHL: CR or PR;
LPL/WM: CR, very good partial response (VGPR), PR, or minor response (MR);
ALL: CR, CRi or unconfirmed complete response (CRu) (for subjects with mediastinal disease), or PR; and
AML: CR, CRi, morphologic leukemia-free state (MLFS), or PR.

CR without measurable residual disease (CR_{MRD-}) is defined as the achievement of ≤1×10⁻⁴ malignant cells in bone marrow (as assessed by flow cytometry) in a subject who meets all other criteria for CR. It is also expected that ADC-A when provided in accordance with the dosing regimens provided herein will lead to improvements in percent change in tumor dimensions (defined as the percent change from baseline in the sum of the products of the diameters (SPD) of index lesions), progression-free survival (PFS) (defined as the interval from the start of study therapy to the earlier of the first documentation of disease progression/relapse or death from any cause), and overall survival (OS) (defined as the interval from the start of study therapy to death from any cause).

The ADC-A dosing regimen may also lead to changes (e.g., increase or decrease) in plasma concentrations of Wnt5a (as assessed by immunoassay), changes (e.g., increase or decrease) in plasma concentrations of circulating ROR1 (as assessed by immunoassay), and alteration in the numbers (e.g., increase or decrease) or activation status (e.g., increase or decrease) of immune cells such as circulating B cells, T cells and natural killer (NK) cells.

### Patient Selection

In the study, the patients may be adult patients who are 18 years or older; have been diagnosed with CLL/SLL, MCL, FL, MZL, DLBCL, RTL, BL, LPL/WM, T cell NHL, ALL, or AML; have been previously treated but have progressed during or relapsed after prior systemic therapy, or are unlikely responsive to established therapies known to provide clinical benefit or have developed an intolerance to established therapies known to provide clinical benefit; and have completed all previous therapy (including surgery, radiotherapy, chemotherapy, immunotherapy, or investigational therapy) for the treatment of cancer ≥1 week before the start of study therapy.

Patients who have ongoing immunosuppressive therapy other than corticosteroids may be excluded from the treatment. At the time of starting study therapy, subjects may be using systemic corticosteroids (at doses of ≤10 mg/day of prednisone or equivalent), or topical, inhaled, or intra-articular corticosteroids. During study therapy, subjects may use systemic, enteric, topical, inhaled, or intra-articular corticosteroids, as required (e.g., for intercurrent medical conditions or antiemetic prophylaxis).

### Premedication

To prevent tumor lysis syndrome (TLS), the patients may be given allopurinol, 100 to 300 mg orally every 8 hours starting ≥24 to 48 hours before the start of study drug therapy. Alternative drugs (e.g., febuxostat) may be substituted, with administration per product labelling. In addition, subjects with hyperuricemia may receive rasburicase, 3 to 4.5 mg by IV infusion. In addition, high-risk subjects may receive rasburicase, 3 to 4.5 mg by IV infusion, administered 3 to 4 hours prior to the first dose of study drug.

If infusion reactions are observed, subjects may be premedicated before ADC-A infusions with an antipyretic and an antihistamine to reduce the incidence and severity of infusion reactions. The regimen may be an oral or IV antipyretic (acetaminophen [paracetamol], 650 to 1,000 mg or equivalent) and an oral or IV antihistamine (cetirizine, 10 mg or equivalent) both given 30 to 60 minutes prior to each ADC-A infusion. A nonsteroidal antiinflammatory drug (NSAID) (ibuprofen, 400 to 800 mg orally or equivalent) may be added or substituted for acetaminophen. A corticosteroid (100 mg of prednisolone or equivalent) as a premedication can be considered, as needed.

### Efficacy Assessments

### Chronic Lymphocytic Leukemia/Small Lymphocytic Lymphoma

Responses will be categorized as complete response without measurable residual disease (CR_{MRD-}), complete response (CR), complete response with incomplete blood count recovery (CRi), partial response (PR), partial response with lymphocytosis (PR-L), stable disease (SD), or progressive disease (PD). In addition, a response category of nonevaluable (NE) is provided for situations in which there is inadequate information to otherwise categorize response status.

The best overall response will be determined. The best overall response is the best response recorded from the start of treatment until PD/recurrence. The screening measurement will be taken as a reference for determinations of response. The nadir measurement will be taken as a reference for PD; this measurement constitutes the smallest measurement recorded, including the screening measurement if this is the smallest measurement. Where imaging data are available, these data will supersede physical examination data in determining tumor status.

(1) Complete Response without Measurable Residual Disease
To satisfy criteria for CR_{MRD-}, all of the following conditions must be attained:
All criteria for CR are met
Flow cytometry of bone marrow aspirate shows malignant cells of ≤1×10⁻⁴

### (2) Complete Response

To satisfy criteria for CR, all of the following conditions must be attained:
No evidence of new disease
ALC in peripheral blood of <4×10⁹/L
Regression of all index nodal masses to normal size ≤15 mm in the LD
Normal spleen and liver size
Regression to normal of all nodal non-index disease and disappearance of all detectable non-nodal, non-index disease
Morphologically negative bone marrow defined as <30% of nucleated cells being lymphoid cells and no lymphoid nodules in a bone marrow sample that is normocellular for age
Peripheral blood meeting all of the following criteria:
ANC>1.5×10⁹/L without exogenous growth factors ≤2 weeks before the relevant blood count assessment;
platelet count >100×10⁹/L without exogenous growth factors or platelet transfusions ≤2 weeks before the relevant blood count assessment; and
hemoglobin >110 g/L (11.0 g/dL) without exogenous growth factors or red blood cell transfusions ≤2 weeks before the relevant blood count assessment.
   (3) Complete Response with Incomplete Count Recovery
   To satisfy criteria for CRi, all criteria for CR are met except ≥1 of the following conditions exists:
ANC≤1.5×10⁹/L or requires exogenous growth factors ≤2 weeks before the relevant blood count assessment to maintain an ANC≥1.5×10⁹/L
Platelet count ≤100×10⁹/L or requires exogenous growth factors or platelet transfusions ≤2 weeks before the relevant blood count assessment to maintain a platelet count ≤100×10⁹/L
Hemoglobin ≤110 g/L (11.0 g/dL) or requires exogenous growth factors or red blood cell transfusions ≤2 weeks before the relevant blood count assessment to maintain a hemoglobin ≥110 g/L (11.0 g/dL)

### (4) Partial Response

To satisfy criteria for a PR, all of the following conditions must be attained:
No evidence of new disease
A change in disease status meeting 2 of the following criteria, with the exception that if only lymphadenopathy is present at screening, only lymphadenopathy must improve to the extent specified below:
   Decrease in peripheral blood ALC by ≥50% from screening
   A decrease by ≥50% from the screening in the SPD of the index nodal lesions
   In a subject with enlargement of the spleen at screening (>120 mm), a ≥50% decrease from screening (minimum decrease of 20 mm) in the enlargement of the spleen in its LVD or to ≤120 mm by imaging
   In a subject with enlargement of the liver at screening (>180 mm), a ≥50% decrease from screening (minimum decrease of 20 mm) in the enlargement of the liver in its LVD or to ≤180 mm by imaging
   A decrease by ≥50% from screening in the CLL/SLL bone marrow infiltrate or in B-lymphoid nodules
No index, splenic, liver, or non-index disease with worsening that meets the criteria for definitive PD
Peripheral blood meeting ≥1 of the following criteria:
   ANC≥1.5×10⁹/L or ≥50% increase over screening without exogenous growth factors (eg, G-CSF)≤2 weeks before the relevant blood count assessment
   Platelet count >100×10⁹/L or ≥50% increase over screening without exogenous growth factors or platelet transfusions ≤2 weeks before the relevant blood count assessment
   Hemoglobin ≥110 g/L (11.0 g/dL) or ≥50% increase over screening without exogenous growth factors (eg, erythropoietin) or red blood cell transfusions ≤2 weeks before the relevant blood count assessment
      (5) Partial Response with Lymphocytosis
      To satisfy criteria for a PR-L, the following conditions must be attained:
No evidence of new disease
All criteria for PR achieved except for the lack of a decrease in peripheral blood ALC by ≥50% from screening

### (6) Stable Disease

To satisfy criteria for SD, the following conditions must be attained:
No evidence of new disease
There is neither sufficient evidence of tumor shrinkage to qualify for PR nor sufficient evidence of tumor growth to qualify for definitive PD

### (7) Progressive Disease

The occurrence of any of the following events indicates definitive PD:
Evidence of any new disease:
   A new node that measures >15 mm in any diameter
   in a subject with a normal spleen LVD (i.e., a LVD of ≤120 mm by imaging) at nadir, a spleen LVD of >140 mm by imaging
   in a subject with a normal liver LVD (i.e., a LVD of ≤180 mm by imaging) at nadir, a liver LVD of >200 mm by imaging
   New non-index disease (eg, effusions, ascites, or other organ abnormalities related to CLL/SLL)
Evidence of worsening of index lesions, spleen or liver, or non-index disease:
   Increase from the nadir by ≥50% from the nadir in the SPD of index lesions
   Increase from the nadir by ≥50% in the LD of an individual node or extranodal mass that now has an LD of >15 mm and an LPD of >10 mm
   In a subject with enlargement of the spleen at screening (>120 mm), a ≥50% increase in splenic enlargement (minimum increase of 20 mm) from nadir
   In a subject with enlargement of the liver at screening (>180 mm), a ≥50% increase from screening (minimum increase of 20 mm) from nadir
   Unequivocal increase in the size of non-index disease (eg, effusions, ascites, or other organ abnormalities related to CLL/SLL)
   Transformation to a more aggressive histology (eg, RTL) as established by lymph node biopsy (with the date of the lymph node biopsy being considered the date of CLL/SLL progression if the subject has no earlier objective documentation of CLL/SLL progression)
Decrease in platelet count or hemoglobin that is attributable to CLL/SLL, is not attributable to an autoimmune phenomenon, and is confirmed by bone marrow biopsy showing an infiltrate of clonal CLL/SLL cells
   The current platelet count is ≤100×10⁹/L and there has been a decrease by ≥50% from baseline
   The current hemoglobin is ≥110 g/L (11.0 g/dL) and there has been a decrease by >20 g/L (2 g/dL) from baseline

### Lymphoma

Responses will be categorized as complete response without measurable residual disease (CR_{MRD-}), complete response (CR), very good partial responses (VGPR; LPL/WM only), partial response (PR), minor response (MR; LPL/WM only), stable disease (SD), or progressive disease (PD). In addition, a response category of nonevaluable (NE) is provided for situations in which there is inadequate information to otherwise categorize response status.

The best overall response will be determined. The best overall response is the best on-treatment response from screening recorded from the start of treatment until PD/recurrence. The screening measurement will be taken as a reference for determinations of response. The nadir measurement will be taken as a reference for PD; this measurement constitutes the smallest measurement recorded, including the screening measurement if this is the smallest measurement. For FDG-avid tumors, metabolic criteria for response by PET-CT will take precedence over anatomic criteria for response by contrast CT when assessing CR.

(1) Complete Response without Measurable Residual Disease
To satisfy criteria for CR_{MRD-}, all of the following conditions must be attained:
All criteria for CR are met
Flow cytometry of a bone marrow aspirate shows malignant cells of ≤1×10⁻⁴

### (2) Complete Response

To satisfy criteria for CR, all of the following conditions must be attained:
No evidence of new disease
Regression of all index nodal lesions to ≤15 mm in the LD
Regression to ≤15 mm of all nodal non-index disease
Disappearance of all detectable extranodal index and non-index disease
Normal spleen size of >130 mm in LVD by imaging studies
If PET is performed, no evidence of residual disease—i.e., score of 1 (no uptake above background), 2 (uptake ≤mediastinum), or 3 (uptake >mediastinum but ≤liver) on the Deauville 5-point scale
Negative for bone marrow involvement by PET for a PET-avid tumor or by morphological assessment of a unilateral core biopsy; if the bone marrow biopsy is indeterminate by morphology, it should be negative by immunohistochemistry
Absence of serum M-protein by SIFE (in subjects with LPL/WM)
   (3) Very Good Partial Response (LPL/WM only)
   To satisfy criteria for VGPR, all of the following conditions must be attained:
All anatomic criteria for CR are met
A ≥90% decrease from baseline in serum M-protein concentration is documented

### (4) Partial Response

To satisfy criteria for PR, all of the following conditions must be attained:
No evidence of new disease
A ≥50% decrease from screening in the SPD of the index nodal and extranodal lesions
No increase from the nadir in the size of non-index disease
In a subject with enlargement of the spleen at screening, a ≥50% decrease from screening (minimum decrease of 20 mm) in the enlargement of the spleen in its longest vertical dimension (LVD) or to ≤130 mm by imaging
If PET performed:
   Typically FDG-avid lymphoma: if no screening PET scan was performed or if the PET scan was positive before therapy, the on-treatment PET is positive in ≥1 previously involved site—i.e., score of 4 (uptake moderately >liver) or score of 5 (uptake markedly >liver) on the Deauville 5-point scale but with reduced uptake compared with screening. If a screening PET was performed and was negative, there is no new PET evidence of disease. Reduced uptake is defined as a ≥25% decrease in the % ΔSUVmax.
   Variably FDG-avid lymphoma/FDG-avidity unknown: if no pretreatment PET scan was performed or if the pretreatment PET scan was negative for lymphoma, CT criteria should be used in assessing the tumor during treatment. If the PET scan was positive before therapy, the on-treatment PET is positive in ≥1 previously involved site.
Persistence of bone marrow involvement in a subject who meets radiographic criteria for CR
A ≥50% but <90% decrease from baseline in serum M-protein concentration (in subjects with LPL/WM)

### (5) Minor Response (LPL/WM Only)

To satisfy criteria for MR, all of the following conditions must be attained:
Anatomic disease criteria for PR or SD are met
A ≥25% but <50% decrease from baseline in serum M-protein concentration is documented

### (6) Stable Disease

To satisfy criteria for SD, all of the following conditions must be attained:
No evidence of new disease
Neither sufficient tumor shrinkage from screening to qualify for PR nor sufficient evidence of tumor growth to qualify for PD
If PET performed, the results show a score of 4 (uptake moderately >liver) or score of 5 (uptake markedly >liver) on the Deauville 5-point scale with no significant change in uptake compared with screening
A <25% decrease and <25% increase from baseline in serum M-protein concentration (in subjects with LPL/WM)

### (7) Progressive Disease

The occurrence of any of the following events indicates PD:
Evidence of any new disease that was not present at screening:
   A new node that measures >15 mm in any diameter
   Reappearance of an extranodal lesion that had resolved (i.e., had previously been assigned a PPD of 0 mm²)
   A new extranodal lesion >10 mm
   New non-index disease (eg, effusions, ascites, or other organ abnormalities) of any size unequivocally attributable to lymphoma (usually requires PET, biopsy, cytology, or other non-radiologic confirmation to confirm disease attributable to lymphoma).
   New FDG-avid foci consistent with lymphoma rather than another etiology (e.g., infection, inflammation). If there is uncertainty regarding the etiology of new lesions, biopsy or interval scan may be considered.
   New or recurrent bone marrow involvement with lymphoma by PET or by bone marrow biopsy if prior PET or bone marrow biopsy performed as part of the study was negative for lymphoma.
Evidence of worsening of nodal or extranodal index lesions:
   Increase from the nadir by ≥50% in the SPD of index lesions
   Evidence of worsening of individual index lymph nodes or nodal masses with an increase from the nadir by ≥50% in the PPD for any individual node if the node now has an LD of >15 mm, an increase by 250% from the nadir PPD, and an increase in LD or SDi from the nadir by:
   ≥5 mm for lesions measuring ≤20 mm (in LD or SDi) or
   ≥10 mm for lesions measuring >20 mm (in LD or SDi)
Unequivocal increase in the size of non-index disease
An increase in splenic enlargement by ≥50% (minimum increase of 20 mm) from nadir in a patient with a spleen LVD of >130 mm by imaging at nadir, or a spleen LVD of >150 mm by imaging in a patient with a spleen LVD of ≤130 mm by imaging at nadir;
Transformation to a more aggressive NHL histology as established by lymph node biopsy
If PET performed, there is a score of 4 (uptake moderately >liver) or score of 5 (uptake markedly >liver) on the Deauville 5-point scale with an increase in uptake compared with the nadir in conjunction with an anatomic increase in lesion size consistent with PD. Increased uptake is defined as a ≥50% increase in the % ΔSUVmax
An increase from the nadir by ≥25% in serum M-protein concentration (in subjects with LPL/WM)

### Acute Lymphoid Leukemia

Responses will be categorized as complete response without measurable residual disease (CR_{MRD-}), complete response (CR), complete response with incomplete blood count recovery (CRi) (including also unconfirmed complete response [CRu] for subjects with mediastinal disease), partial response (PR), stable disease (SD), treatment failure (TF) or disease recurrence or progression (DRP). In addition, a response category of nonevaluable (NE) is provided for situations in which there is inadequate information to otherwise categorize response status.

The best overall response will be determined. The best overall response is the best on-treatment response from baseline recorded from the start of treatment until DRP. The baseline measurement will be taken as a reference for determinations of response. The nadir measurement will be taken as a reference for DRP; the best on-study measurement constitutes the measurement with the least tumor involvement, including the baseline measurement if this is the measurement meeting this criterion.

(1) Complete Response without Measurable Residual Disease
To satisfy criteria for CR_{MRD-}, all of the following conditions must be attained:
All criteria for CR are met
Flow cytometry of a bone marrow aspirate shows malignant cells of ≤1×10⁻⁴

### (2) Complete Response

To satisfy criteria for CR, all of the following conditions must be attained:
Leukemia disease status meeting all of the following requirements:
   <5% bone marrow blasts (based on a bone marrow aspirate/biopsy sample with ≥200 nucleated cells and the presence of bone marrow spicules)
   No blasts in the peripheral blood
   No extramedullary disease (including lymphadenopathy, splenomegaly, skin/gum infiltration, testicular mass, and no central nervous system involvement [i.e., attainment of CNS-1 status {no blasts in the cerebrospinal fluid}])
Peripheral blood meeting all of the following requirements:
   ANC≥1.0×10⁹/L
   Platelet count ≥100×10⁹/L
Any mediastinal enlargement shows complete resolution as documented radiographically
   (3) Complete Response with Incomplete Blood Count Recovery and/or Complete Response Unconfirmed
   To satisfy criteria for CRi/CRu, all of the following conditions must be attained:
Leukemia disease status meeting all of the following requirements:
   <5% bone marrow blasts (based on a bone marrow aspirate/biopsy sample with ≥200 nucleated cells and the presence of bone marrow spicules)
   No blasts in the peripheral blood
   No extramedullary disease (including lymphadenopathy, splenomegaly, skin/gum infiltration, testicular mass, and no central nervous system involvement [i.e., attainment of CNS-1 status {no blasts in the cerebrospinal fluid}])
Peripheral blood meeting any of the following requirements:
   ANC<1.0×10⁹/L
   Platelet count <100×10⁹/L
Any mediastinal enlargement has regressed by ≥75% in the sum of the products of the perpendicular diameters (SPD) as documented radiographically

### (4) Partial Response

To satisfy criteria for PR, all of the following conditions must be attained:
Leukemia disease status meeting either of the following requirements:
   A ≥50% decrease in bone marrow blasts to 5% to 25% (inclusive) (based on a bone marrow aspirate/biopsy sample with ≥200 nucleated cells and the presence of bone marrow spicules)
   No blasts in the peripheral blood
   No new or worsening extramedullary disease (including lymphadenopathy, splenomegaly, skin/gum infiltration, testicular mass, or central nervous system involvement [eg, CNS-1 status {no blasts in the cerebrospinal fluid} has not transitioned to CNS-2 status {WBC<5×10⁹/L with presence of blasts in the cerebrospinal fluid} or to CNS-3 status {WBC≥5×10⁹/L with presence of blasts in the cerebrospinal fluid} or to development of facial nerve palsy, brain/eye involvement, or hypothalamic syndrome)
Peripheral blood meeting all of the following requirements:
   ANC≥1.0×10⁹/L
   Platelet count ≥100×10⁹/L
Any mediastinal enlargement has regressed by ≥50% in the SPD as documented radiographically

### (5) Stable Disease/Treatment Failure

To satisfy criteria for SD, all of the following conditions must be attained:
Neither sufficient ALL improvement from baseline to qualify for PR nor sufficient evidence of ALL worsening to qualify for DRP
No new or worsening extramedullary disease (including lymphadenopathy, splenomegaly, skin/gum infiltration, testicular mass, or central nervous system involvement [e.g., CNS-1 status {no blasts in the cerebrospinal fluid} has not transitioned to CNS-2 status {WBC<5×10⁹/L with presence of blasts in the cerebrospinal fluid} or to CNS-3 status {WBC≥5×10⁹/L with presence of blasts in the cerebrospinal fluid} or to development of facial nerve palsy, brain/eye involvement, or hypothalamic syndrome)
No development of new mediastinal enlargement and no increase in the SPD of existing mediastinal enlargement by >25%
   A subject without DRP who does not qualify for a CR_{MRD-}, CR, CRi/CRu, or PR by 18 weeks (for Schedules 1 and 2) or 16 weeks (for Schedule 3) from the start of study therapy will be considered to have TF.

### (6) Disease Recurrence or Progression

The occurrence of any of the following events indicates DRP:
Reappearance of bone marrow blasts (to >5%) in a subject who had experienced a CR
Reappearance of blasts in the peripheral blood in a subject who had experienced a CR
A ≥25% increase in bone marrow blasts to ≥20% (based on a bone marrow aspirate/biopsy sample with ≥200 nucleated cells and the presence of bone marrow spicules)
A ≥25% increase in blasts in the peripheral blood to >1×10⁹/L.
Development of new or worsening existing extramedullary disease (involving lymphadenopathy, splenomegaly, skin/gum infiltration, testicular mass, or CNS involvement [e.g., CNS-1 status {no blasts in the cerebrospinal fluid} has transitioned to CNS-2 status {WBC<5×10⁹/L with presence of blasts in the cerebrospinal fluid} or to CNS-3 status {WBC≥5×10⁹/L with presence of blasts in the cerebrospinal fluid} or to development of facial nerve palsy, brain/eye involvement, or hypothalamic syndrome)
Development of new mediastinal enlargement or increase in the SPD of existing mediastinal enlargement by 225%

### Acute Myeloid Leukemia

Responses will be categorized as complete response without measurable residual disease (CR_{MRD-}), complete response (CR), complete response with incomplete blood count recovery (CRi), morphologic leukemia-free state (MLFS), partial response (PR), stable disease (SD), treatment failure (TF), or disease recurrence or progression (DRP). In addition, a response category of nonevaluable (NE) is provided for situations in which there is inadequate information to otherwise categorize response status.

The best overall response will be determined. The best overall response is the best on-treatment response from baseline recorded from the start of treatment until DRP or TF. The baseline status will be taken as a reference for determinations of response. The best on-study measurement will be taken as a reference for DRP; the best on-study measurement constitutes the measurement with the least tumor involvement, including the baseline measurement if this is the measurement meeting this criterion.

(1) Complete Response without Measurable Residual Disease
To satisfy criteria for CR_{MRD-}, all of the following conditions must be attained:
All criteria for CR are met
Flow cytometry of a bone marrow aspirate shows malignant cells of ≤1×10⁻⁴

### (2) Complete Response

To satisfy criteria for CR, all of the following conditions must be attained:
Leukemia disease status meeting all of the following requirements:
   <5% bone marrow blasts (based on a bone marrow aspirate/biopsy sample with ≥200 nucleated cells and the presence of bone marrow spicules)
   No blasts in the peripheral blood
   No blasts with Auer rods
   No extramedullary disease
Peripheral blood meeting both of the following requirements:
   ANC≥1.0×10⁹/L
   Platelet count ≥100×10⁹/L
      (3) Complete Response with Incomplete Blood Count Recovery
      To satisfy criteria for CRi, all of the following conditions must be attained:
Leukemia disease status meeting all of the following requirements:
   <5% bone marrow blasts (based on a bone marrow aspirate/biopsy sample with ≥200 nucleated cells and the presence of bone marrow spicules)
   No blasts in the peripheral blood
   No blasts with Auer rods
   No extramedullary disease
Peripheral blood meeting only 1 of the following criteria:
   ANC≥1.0×10⁹/L
Platelet count ≥100×10⁹/L

### (4) Morphologic Leukemia-Free State

To satisfy criteria for MLFS, all of the following conditions must be attained:
Leukemia disease status meeting all of the following requirements:
   <5% bone marrow blasts (based on a bone marrow aspirate/biopsy sample with ≥200 nucleated cells, ≥10% cellularity, and the presence of bone marrow spicules)
   No blasts in the peripheral blood
   No blasts with Auer rods
   No extramedullary disease
Peripheral blood meeting both of the following criteria:
   ANC<1.0×10⁹/L
   Platelet count <100×10⁹/L

### (5) Partial Response

To satisfy criteria for PR, all of the following conditions must be attained:
Leukemia disease status meeting either of the following requirements:
   A ≥50% decrease in bone marrow blasts to 5% to 25% (inclusive) (based on a bone marrow aspirate/biopsy sample with ≥200 nucleated cells and the presence of bone marrow spicules)
   <5% bone marrow blasts but with Auer rods present (based on a bone marrow aspirate/biopsy sample with ≥200 nucleated cells and the presence of bone marrow spicules)
   No blasts in the peripheral blood
   No new or worsening extramedullary disease
Peripheral blood meeting both of the following criteria:
   ANC≥1.0×10⁹/L
   Platelet count ≥100×10⁹/L

### (6) Stable Disease/Treatment Failure

To satisfy criteria for SD, all of the following conditions must be attained:
Neither sufficient AML improvement from baseline to qualify for CR_{MRD-}, CR, CRi, MLFS, or PR, nor sufficient evidence of AML worsening to qualify for DRP
No new or worsening extramedullary disease
   A subject without DRP who does not qualify for a CR_{MRD-}, CR, CRi, MLFS, or PR by 18 weeks (for Schedules 1 and 2) or 16 weeks (for Schedule 3) from the start of study therapy will be considered to have TF.

### (7) Disease Relapse or Progression

The occurrence of any of the following events indicates DRP:
Reappearance of bone marrow blasts to >5% in a subject who had experienced a CR_{MRD-}, CR, CRi, or MLFS
Reappearance of blasts in the peripheral blood in a subject who had experienced a CR_{MRD-}, CR, CRi, MLFS, or PR
An absolute 20% increase in bone marrow blasts to >25% (based on a bone marrow aspirate/biopsy sample with ≥200 nucleated cells and the presence of bone marrow spicules) in a subject who had experienced a PR
An absolute 20% increase in peripheral blasts to >25% in a subject who had experienced a PR
Development of new extramedullary disease or worsening of existing extramedullary disease Reoccurrence or worsening of MRD, as assessed by flow cytometry, will not be considered in the definition of DRP, but will be recorded.

### Laboratory and Other Assessments

Samples to be obtained and parameters to be analyzed are indicated in Table 4.

TABLE 4
Laboratory and Other Parameters to Be Assessed

| | |
|---|---|
| Test or Procedure | Parameters |

Safety (analyzed at a local clinical laboratory)

| | |
|---|---|
| Urinalysis | Dipstick: specific gravity, pH, protein, glucose, ketones, bilirubin, |
| | urobilinogen, blood, nitrite, leukocyte esterase |
| | Microscopy: White blood cells, red blood cells, epithelial cells, |
| | bacteria, casts, crystals |
| Serum virology | Serum HIV antibody |
| | Serum HBsAg antibody, HBc antibody (or serum HBV DNA by |
| | PCR) |
| | Serum HCV antibody (or serum HCV RNA by PCR) |
| Serum pregnancy test | Serum β-HCG |
| Serum chemistry | Sodium, potassium, chloride, bicarbonate, BUN, creatinine, |
| | glucose, calcium, phosphorus, magnesium, total protein, albumin, |
| | ALT, AST, ALP, CK, LDH, total bilirubin, uric acid, amylase, |
| | lipase |
| Hematology | Hematocrit, hemoglobin, erythrocyte count |
| | Absolute counts of leukocytes, neutrophils, lymphocytes, |
| | monocytes, eosinophils, basophils |
| | Platelet count |
| Coagulation | PT |
| | aPTT |

Pharmacokinetics (analyzed at a contract laboratory)

| | |
|---|---|
| ADC-A and MMAE | Plasma ADC-A concentrations (as assessed by a validated |
| pharmacokinetics | bioanalytical method), including: |
| | Total ADC-A, defined as the complete antibody-linker-MMAE |
| | conjugate (considering the mean DAR to derive the total ADC-A |
| | value) |
| | Total antibody, defined as total ADC-A plus any antibody that is |
| | not conjugated to MMAE |
| | Plasma MMAE concentrations (as assessed by a validated |
| | bioanalytical method) |
| | Retention of plasma for potential metabolite analyses |

Immunogenicity (analyzed at a contract laboratory)

| | |
|---|---|
| Serum for ADC-A | Titers and neutralizing capacity of ADA (as assessed by a validated |
| immunogenicity | immunoassay) |
| | Retention of serum for alternative safety parameter analysis |

Pharmacodynamics (analyzed at contract laboratories)

| | |
|---|---|
| Plasma | Plasma Wnt5 concentrations (as measured using immunoassay |
| pharmacodynamics | methods) |
| | Plasma ROR1 concentrations (as measured using immunoassay |
| | methods) |
| | Chemokines and cytokines, potentially including (but not limited |
| | to): CCL2, CCL3, CCL4, CCL7, CCL17, CCL19, CCL21, CCL22, |
| | CXCL12, CXCL13, CD40 ligand, and TNFα (as measured using |
| | immunoassay methods) |
| | Retention of plasma for potential additional cytokine/chemokine |
| | analyses, ADC-A concentration analysis, or alternative safety |
| | parameter analysis |
| Blood for ROR1/other | Numbers of circulating tumor cells with expression of ROR1 and |
| markers (CLL/SLL, | other markers of proliferation/apoptosis (e.g., Ki67, caspase) (as |
| MCL, T cell NHL, | measured using flow cytometry) |
| ALL, and AML) | |
| Blood for T-, B-, and | Numbers of circulating T cell, B cell, NK cell, and monocyte |
| NK-cell profiling | subsets (as measured using flow cytometry) |

Disease-Related Biomarkers (analyzed at contract laboratories)

| | |
|---|---|
| Tumor (from | Baseline tumor protein expression of ROR1 (as assessed by |
| peripheral blood, bone | immunohistochemistry) |
| marrow, or tumor | Baseline tumor mutational, gene expression, and/or protein gene |
| biopsy) for | expression profiling (as assessed by NanoString technology) |
| ROR1/other profiling | Cell of origin (DLBCL only) (as assessed by NanoString |
| | Lymph2Cx) |

Disease-Related Efficacy (analyzed at a contract laboratory or a local clinical laboratory
(for M protein))

| | |
|---|---|
| Bone marrow biopsy | Bone marrow biopsy for analysis of hematological cancer disease |
| and aspirate (in | status (as assessed by hematoxylin and eosin, |
| subjects with CLL/SLL | immunohistochemistry and/or FISH [as needed for ambiguous |
| or NHL) | disease analysis]) |
| | Bone marrow aspirate for MRD status (as assessed by flow |
| | cytometry) |
| Peripheral blood | Peripheral blood smears for analysis of ALL/AML disease status |
| smears and bone | (blast percentage, presence of Auer rods [AML] (as assessed by |
| marrow biopsy and | Wright stain) |
| aspirate (in subjects | Bone marrow aspirate for analysis of ALL/AML disease status |
| with ALL or AML) | (blast percentage, presence of Auer rods [AML]) (as assessed by |
| | Wright stain) and MRD status (as assessed by flow cytometry) |
| | Bone marrow biopsy for analysis of hematological cancer disease |
| | status (as assessed by hematoxylin and eosin, |
| | immunohistochemistry, and/or FISH [as needed for ambiguous |
| | disease analysis]) |
| Serum for M-protein | Serum immunoglobulin concentrations (as assessed by SPEP and |
| (in subjects with | SIFE) |
| LPL/WM) | |

Disease-Related Efficacy (analyzed at a contract radiographic imaging facility)

| | |
|---|---|
| Radiology examination | Imaging of neck, chest abdomen, and pelvis (by CT, PET/CT, or |
| | MRI as appropriate for disease type and tumor status). |

Other (analyzed at the site - except ECG, which is analyzed at a contract cardiology
facility)

| | |
|---|---|
| Body weight/height | Weight in kilograms, height in centimeters |
| Body temperature | Temperature in degrees Celsius |
| Blood pressure | Diastolic and systolic blood pressure in mm Hg |
| Oxygen saturation | % saturation |
| 12-lead ECG | Heart rate, cardiac intervals, wave form abnormalities, ectopy |

Abbreviations: ADA = antidrug antibodies, ALL = acute lymphoid leukemia, ALP = alkaline phosphatase, ALT = alanine aminotransferase, AML = acute myeloid leukemia, aPTT = activated partial thromboplastin time, AST = aspartate aminotransferase, BUN = blood urea nitrogen, CCL = chemokine (C—C motif) ligand, CD = cluster of differentiation, CK = creatine kinase, CLL = chronic lymphocytic leukemia, CT = computed tomography, CXCL = chemokine (C-X-C motif) ligand, DAR = drug-antibody ratio, DLBCL = diffuse large B-cell lymphoma, DNA = deoxyribonucleic acid, ECG = electrocardiogram, FDG = fluorodeoxyglucose, FISH = fluorescent in situ hybridization, HBc antibody = anti-hepatitis B core antibody, HBsAg = hepatitis B surface antigen, HBV = hepatitis B virus, HCV = hepatitis C virus, HIV = human immunodeficiency virus, IFNγ = interferon-γ, IL = interleukin, LDH = lactate dehydrogenase, LPL/WM = lymphoplasmacytoid lymphoma/Waldenström macroglobulinemia, M-protein = monoclonal immunoglobulin M protein, MMAE = monomethyl auristatin E, MRD = measurable residual disease, MRI = magnetic resonance imaging, NGS = next-generation sequencing, NK = natural killer (cells), PET = positron emission tomography, PCR = polymerase chain reaction, PT = prothrombin time, RNA = ribonucleic acid, ROR1 = receptor tyrosine kinase-like orphan receptor-1, SIFE = serum immunofixation electrophoresis, SPEP = serum protein electrophoresis, TNFα = tumor necrosis factor, β-HCG = β-human chorionic gonadotropin

Grading of adverse event severity, as applied herein, is described in Table 5 below.

TABLE 5
Grading of Adverse Event Severity

| Grade | Adjective | Description |
|---|---|---|
| Grade 1 | Mild | Sign or symptom is present, but it is easily tolerated, |
| | | is not expected to have a clinically significant effect |
| | | on the subject's overall health and well-being, does |
| | | not interfere with the subject's usual function, and is |
| | | not likely to require medical attention. |
| Grade 2 | Mod- | Sign or symptom causes interference with usual |
| | erate | activity or affects clinical status and may require |
| | | medical intervention. |
| Grade 3 | Severe | Sign or symptom is incapacitating or significantly |
| | | affects clinical status and likely requires medical |
| | | intervention and/or close follow-up. |
| Grade 4 | Life- | Sign or symptom results in a potential threat to life. |
| | threat- | |
| | ening | |
| Grade 5 | Fatal | Sign or symptom results in death. |

### Results

### I. Safety

Data have been obtained from 25 subjects with hematological malignancies, including 12 subjects with MCL, 7 subjects with CLL/SLL, 2 subjects with DLBCL, 2 h subjects with FL, 1 subject with MZL, and 1 subject with RTL. Subjects were heavily pretreated with a median (range) of 4 (1-23) prior systemic chemotherapy regimens, including hematopoietic stem cell transplantation (HSCT) in 4 subjects and chimeric antigen receptor (CAR)-T-cell or natural killer (NK) therapy in 3 subjects.

96 doses of ADC-A were administered, including 1 at the 0.5 mg/kg dose level, 9 at the 1.0 mg/kg dose level, 18 at the 1.5 mg/kg dose level, 46 at the 2.25 mg/kg dose level, and 22 at the 2.5 mg/kg dose level. The number of cycles of therapy received ranged from 1 to 10. All subjects received ADC-A on the Q1/3W schedule of administration (Table 2).

Treatment with ADC-A was generally well-tolerated, with neutropenia being the primary acute toxicity. No DLTs were observed at doses of 0.5, 1.0, and 1.5 mg/kg. A DLT of Grade 4 neutropenia in C1 was noted in 1 of 7 subjects at ADC-A 2.25 mg/kg. In addition, 1 subject receiving ADC-A 2.25 mg/kg experienced Grade 3 neutropenia in C1, and 1 subject receiving ADC-A 2.25 mg/kg experienced Grade 4 neutropenia in C2. In each of these 3 cases, neutropenia was observed on approximately Day 15 of the cycle. Neutropenia was responsive to granulocyte colony-stimulating factor (G-CSF) given reactively or as secondary prophylaxis. No neutropenic fever or infection occurred. A subject starting at ADC-A 2.5 mg/kg experienced Grade 4 thrombocytopenia in C1; however, this subject had a history of thrombocytopenia, including Grade 2 thrombocytopenia at baseline, her post-baseline platelet abnormalities were not clearly drug related, and she continued with C2 and C3 therapy at ADC-A 2.5 mg/kg.

One subject had Grade 2 neuropathy following administration of 5 cycles of ADC-A 2.25 mg/kg; treatment was delayed for 1 cycle and therapy was resumed at a dose of 1.5 mg/kg. Other adverse events, laboratory abnormalities, and ECG findings were low-grade, did not appear to be dose- or exposure-dependent, and likely had resulted from the underlying cancer, comorbid conditions, intercurrent illnesses, or concomitant medications. No infusion reactions or tumor lysis syndrome were observed.

### II. Pharmacokinetics

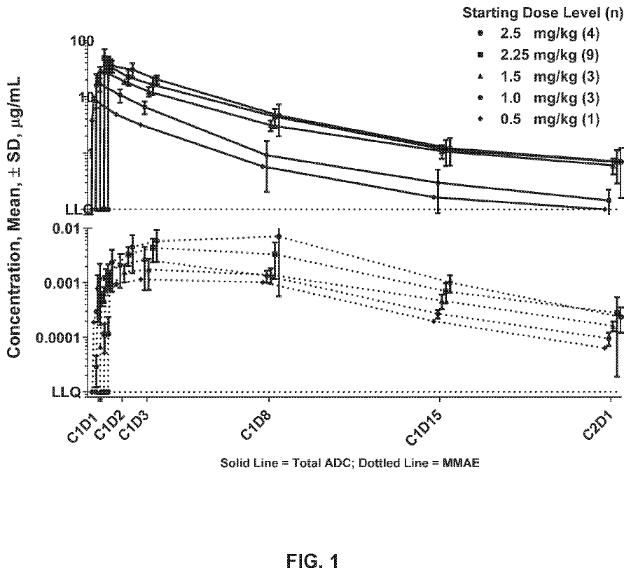

Plasma concentrations of total ADC-A and MMAE over time for 16 patients dosed with ADC-A are shown in FIG. 1. Mean Tₘₐₓ occurred shortly after the end of the infusion for the ADC (0.5 to 2 hours from the start of the 30-minute IV infusion) and at 48 to 89 hours post dose for MMAE. The corresponding pharmacokinetic (PK) parameters are shown in Table 6.

TABLE 6
ADC-A Pharmacokinetic Parameters

| | | | | | | | | | | Ratio |
|---|---|---|---|---|---|---|---|---|---|---|
| | | | | | | | | | | ADC/ |
| | Parameter | Tₘₐₓ | Cₘₐₓ | | AUCₗₐₛₜ | | Vz | CL | t_{1/2} | Antibody |
| Dose | Units | hour | μg/mL | Cₘₐₓ/Dose | h*μg/mL | AUCₗₐₛₜ/Dose | mL/kg | mL/min/kg | days | [a] |
| 0.5 | Antibody | 2 | 12.4 | 25 | 1280 | 2560 | 49.4 | 0.00589 | 4.0 | 0.43 |
| mg/kg | ADC | 2 | 9.6 | 19 | 547 | 1094 | 82.4 | 0.01480 | 2.7 | |
| (n = 1) | MMAE | 48 | 0.00115 | 0.0023 | 0.271 | 0.5420 | | | | |
| 1.0 | Antibody | 2.2 | 20.1 | 20 | 1863 | 1863 | 49.2 | 0.00974 | 3.2 | 0.53 |
| mg/kg | ADC | 2 | 20.6 | 21 | 995 | 995 | 69.0 | 0.01700 | 2.1 | |
| (n = 3) | MMAE | 48 | 0.00266 | 0.0027 | 0.467 | 0.4673 | | | | |
| 1.5 | Antibody | 3.3 | 29.5 | 20 | 3890 | 2593 | 66.0 | 0.00525 | 6.2 | 0.61 |
| mg/kg | ADC | 1.7 | 31.3 | 21 | 2383 | 1589 | 63.4 | 0.01023 | 3.0 | |
| (n = 3) | MMAE | 88.7 | 0.00166 | 0.0011 | 0.390 | 0.2600 | | | | |
| 2.25 | Antibody | 0.8 | 42.5 | 19 | 4020 | 1787 | 70.8 | 0.00935 | 4.4 | 0.66 |
| mg/kg | ADC | 0.5 | 49.3 | 22 | 2660 | 1182 | 70.9 | 0.01621 | 2.4 | |
| (n = 9) | MMAE | 75.1 | 0.00443 | 0.0020 | 0.698 | 0.3102 | | | | |
| 2.5 | Antibody | 2.1 | 48.025 | 19 | 4490.5 | 1796.2 | 68.0 | 0.00747 | 4.6 | 0.64 |
| mg/kg | ADC | 1.3 | 43.25 | 17 | 2967.5 | 1187 | 67.0 | 0.01297 | 2.6 | |
| (n = 4) | MMAE | 78.5 | 0.00670 | 0.0027 | 0.99475 | 0.3979 | | | | |

a AUCₗₐₛₜ for total ADC divided by AUCₗₐₛₜ for total antibody
Abbreviations:
ADC = antibody-drug conjugate, AUCₗₐₛₜ = area under the concentration-time curve up to the last measurable concentration, CL = clearance, Cₘₐₓ = maximum concentration, MMAE = monomethyl auristatin E, SD = standard deviation, t_{1/2} = half-life, Tₘₐₓ = time to maximum concentration, Vz = volume of distribution

Increases in Cₘₐₓ and AUC for were generally dose-proportional for ADC-A and somewhat less than dose-proportional for MMAE. Mean tin values for ADC-A ranged from 2.1 to 3.0 days, independent of the dose administered.

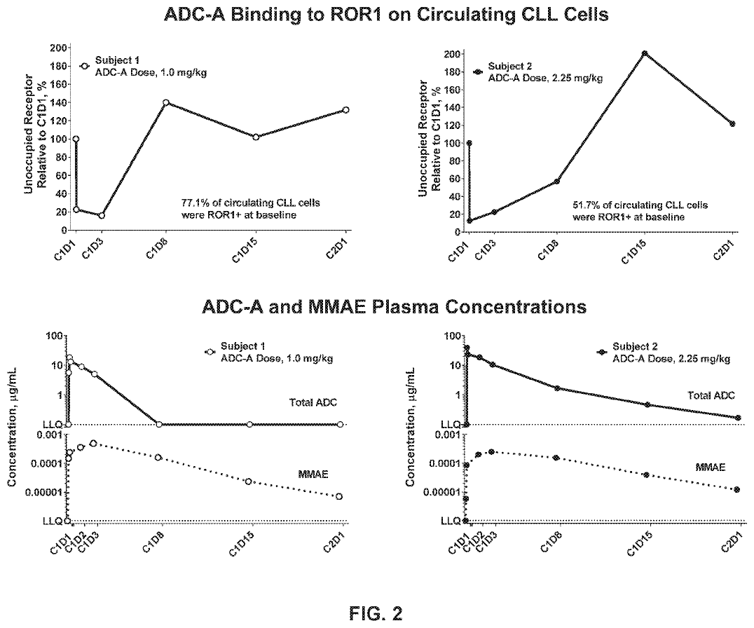

Pharmacodynamic data from subjects with CLL have shown concentration- and time-dependent ADC-A occupancy of ROR1 receptors in circulating CLL cells (FIG. 2). Cells were isolated by Ficoll, stained with LIVE/Dead reagent, and then stained with CD19, CD5, UC961-PE, and 4A5 AlexaFluor647. At respective ADC-A doses of 1.0 and 2.25 mg/kg, only 22.7% and 12.8% of ROR1 receptors were unoccupied by the end of the 30-minute IV infusion. Unoccupied receptors showed a time-dependent return toward baseline that corresponded with simultaneous decreases in ADC-A plasma concentrations. At the 2.25 mg/kg dose level, target coverage appeared to diminish by Day 8 and was lost by Day 15, consistent with declining plasma ADC exposure.

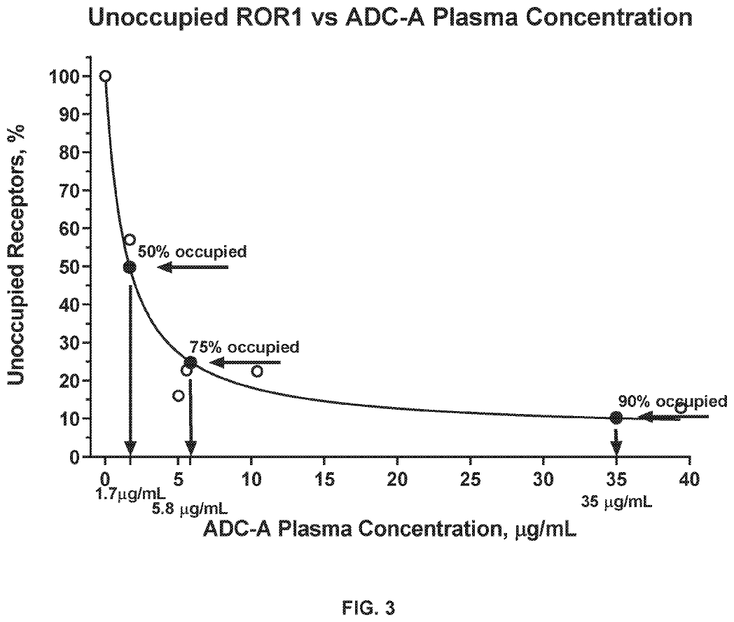

The correlation of unoccupied ROR1 receptors with ADC-A plasma concentrations, as shown in FIG. 3, allows for establishment of target plasma concentration values. Maintaining 50% receptor occupancy (50% unoccupied receptors) requires an ADC-A plasma concentration of 1.7 μg/mL. Maintaining 75% receptor occupancy (25% unoccupied receptors) requires an ADC-A plasma concentration of 5.8 μg/mL. Maintaining 90% receptor occupancy (10% unoccupied receptors) requires an ADC-A plasma concentration of 35 μg/mL.

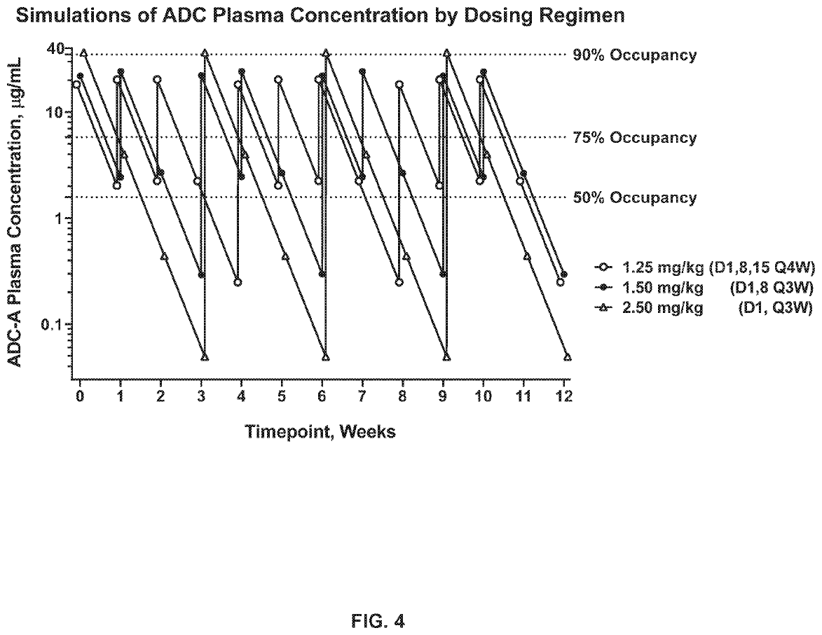

Pharmacokinetic simulations were run to further explore potential ADC-A dosing regimens. The simulations indicate that weekly administration of ADC-A may be more effective than administration once every three weeks (FIG. 4 and Table 7). A weekly dosing regimen may provide more continuous ADC-A exposure and ROR1 target occupancy while allowing myeloid recovery before the next treatment cycle, and may be useful for an induction regimen (e.g., Q2/3W or Q3/4W) followed by a maintenance regimen with less frequent administration (e.g., Q1/3W).

TABLE 7
Maintenance of ROR1 Occupancy over 12 Weeks

| | | | | | Proportion of Time (%) | | |
|---|---|---|---|---|---|---|---|
| ADC-A | | | | Total | Through 12 Weeks | | |
| Dose per | | | Total | Doses per | Achieving Designated | | |
| Infusion, | | Cycles, | Duration, | 12-Week | ROR1 Occupancy | | |
| mg/kg | Schedule | n | Weeks | Period, n | ≥50% | ≥75% | ≥90% |
| 1.25 | D1, D8, D15 Q4W | 3 | 12 | 9 | 78.1 | 41.5 | 0.0 |
| 1.50 | D1, D8 Q3W | 4 | 12 | 8 | 73.4 | 41.7 | 0.0 |
| 2.50 | D1 Q3W | 4 | 12 | 4 | 46.4 | 27.8 | 0.6 |

### II. Antitumor Response

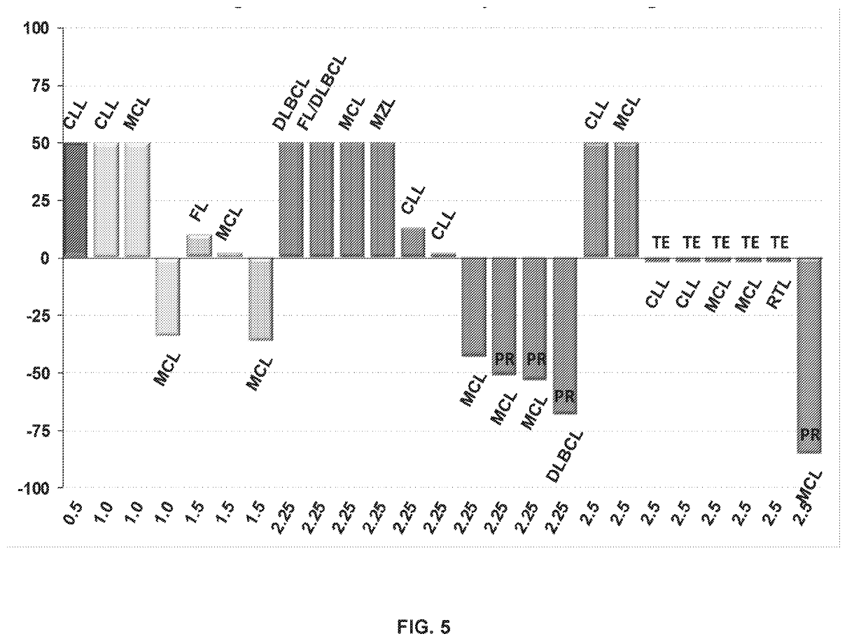

Antitumor activity was observed in heavily pretreated subjects with MCL (including three partial responses) and DLBCL (including one partial response) (FIG. 5) upon treatment with ADC-A using the Q1/3W dosing regimen.

One subject with MCL displayed extensive preexisting disease, with lesions in the palate, neck, chest, abdomen, and pelvis. The subject had received heavy prior therapy with R-hyper-CVAD with R/methotrexate/cytarabine, rituximab, ibrutinib, daratumumab, lenalidomide-rituximab, and radiotherapy. The subject showed evidence of an objective tumor response to treatment with 2.25 mg/kg of ADC-A over three cycles. All measured nodal groups showed reductions in tumor dimensions, with a 53% decrease in SPD (sum of the products of the perpendicular diameters). Further, a palate lesion also decreased in size and tissue infiltration. The magnitude of the response appears to qualify as a partial response (PR). The subject did not display signs of any drug-related hematologic or nonhematologic toxicity.

Another subject with MCL displayed both a palate mass and extranodal disease, and had received heavy prior therapy with rituximab, rituximab/bortezomib, R-CHOP (rituximab, cyclophosphamide, doxorubicin, vincristine, and prednisone), BR (bendamustine and rituximab, R-hyper-CVAD with intrathecal prophylaxis, ibrutinib, and mosunetuzumab. The subject showed evidence of an objective tumor response to treatment with 2.5 mg/kg of ADC-A over three cycles. The subject reported that his activity had increased and fatigue had largely resolved; further, the palate lesion decreased in size by 85%. The magnitude of the response appears to qualify as a partial response (PR).

Another subject with MCL displayed orbit lesions and extranodal disease, and had received heavy prior therapy with R-CHOP, rituximab, ibrutinib-rituximab, and rituximab-BEAM (carmustine, cytarabine, etoposide, and melphalan). The subject showed evidence of an objective tumor response to treatment with 2.5 mg/kg of ADC-A over six cycles, with a 51% decrease in SPD for tumor lesions. The magnitude of the response appears to qualify as a partial response (PR).

One subject with DLBCL displayed extranodal disease, and had received heavy prior therapy with R-CHOP, R-ESHAP (rituximab, etoposide, methylprednisolone, cytarabine, and cisplatin), R-GEMOX (rituximab, gemcitabine, and oxaliplatin), BEAM plus autologous transplant, pinatuzumab-rituximab, bendamustine-rituximab, and CAR-T cells with fludarabine conditioning. The subject showed evidence of an objective tumor response to treatment with ADC-A over six cycles (2.25 mg/kg in Cycle 1, a reduced dose in Cycle 2, 2.25 mg/kg in Cycles 3-5, and 2.5 mg/kg in Cycles 6 and 7). Both measured nodal groups showed reductions in tumor dimensions, with a 68% decrease in SPD. The magnitude of the response appears to qualify as a partial response (PR).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

The amino acid sequences described herein are listed in Table 8 below.

TABLE 8
List of Sequences

| SEQ | Description | SEQUENCE |
|---|---|---|
| 1 | Ab1 heavy | QVQLQESGPGLVKPS |
| | chain | QTLSLTCTVSGYAFT |
| | | AYNIHWVRQAPGQGL |
| | | EWMGSFDPYDGGSSY |
| | | NQKFKDRLTISKDTS |
| | | KNQVVLTMTNMDPVD |
| | | TATYYCARGWYYFDY |
| | | WGHGTLVTVSSASTK |
| | | GPSVFPLAPSSKSTS |
| | | GGTAALGCLVKDYFP |
| | | EPVTVSWNSGALTSG |
| | | VHTFPAVLQSSGLYS |
| | | LSSVVTVPSSSLGTQ |
| | | TYICNVNHKPSNTKV |
| | | DKKVEPKSCDKTHTC |
| | | PPCPAPELLGGPSVF |
| | | LFPPKPKDTLMISRT |
| | | PEVTCVVVDVSHEDP |
| | | EVKFNWYVDGVEVHN |
| | | AKTKPREEQYNSTYR |
| | | VVSVLTVLHQDWLNG |
| | | KEYKCKVSNKALPAP |
| | | IEKTISKAKGQPREP |
| | | QVYTLPPSRDELTKN |
| | | QVSLTCLVKGFYPSD |
| | | IAVEWESN |
| | | GQPENNYKTTPPVLD |
| | | SDGSFFLYSKLTVDK |
| | | SRWQQGNVFSCSVMH |
| | | EALHNHYTQKSLSLS |
| | | PGK |
| 2 | Ab1 light | DIVMTQTPLSLPVTP |
| | chain | GEPASISCRASKSIS |
| | | KYLAWYQQKPGQAPR |
| | | LLIYSGSTLQSGIPP |
| | | RFSGSGYGTDFTLTI |
| | | NNIESEDAAYYFCQQ |
| | | HDESPYTFGEGTKVE |
| | | IKRTVAAPSVFIFPP |
| | | SDEQLKSGTASVVCL |
| | | LNNFYPREAKVQWKV |
| | | DNALQSGNSQESVTE |
| | | QDSKDSTYSLSSTLT |
| | | LSKADYEKHKVYACE |
| | | VTHQGLSSPVTKSFN |
| | | RGEC |
| 3 | Abl VH | QVQLQESGPGLVKPS |
| | | QTLSLTCTVSGYAFT |
| | | AYNIHWVRQAPGQGL |
| | | EWMGSFDPYDGGSSY |
| | | NQKFKDRLTISKDTS |
| | | KNQVVLTMTNMDPVD |
| | | TATYYCARGWYYFDY |
| | | WGHGTLVTVSS |
| 4 | Abl VL | DIVMTQTPLSLPVTP |
| | | GEPASISCRASKSIS |
| | | KYLAWYQQKPGQAPR |
| | | LLIYSGSTLQSGIPP |
| | | RFSGSGYGTDFTLTI |
| | | NNIESEDAAYYFCQQ |
| | | HDESPYTFGEGTKVE |
| | | IK |
| 5 | Ab1 HCDR1 | GYAFTAYN |
| 6 | Ab1 HCDR2 | FDPYDGGS |
| 7 | Ab1 HCDR3 | GWYYFDY |
| 8 | Ab1 LCDR1 | KSISKY |
| 9 | Ab1 LCDR2 | SGS |
| 10 | Ab1 LCDR3 | QQHDESPY |

*SEQ: SEQ ID NO.

1. A method of treating a cancer patient using an immunoconjugate comprising an antibody conjugated to a drug moiety, wherein the immunoconjugate is ADC-A, and has the structure shown in Formula (I) below: 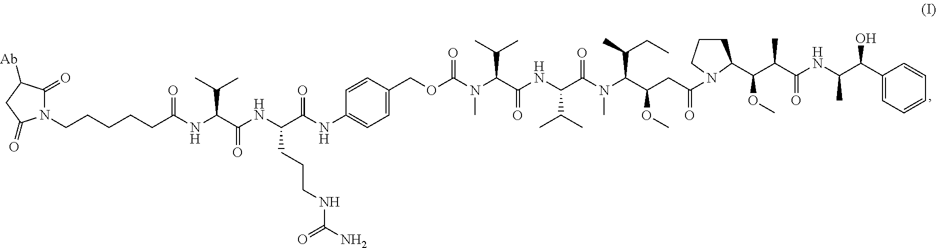
wherein Ab in Formula (I) is the antibody, and wherein the heavy chain and light chain of the antibody comprise the amino acid sequences of SEQ ID NOs: 1 and 2, respectively; and
wherein the immunoconjugate is administered to the patient at a dose of 0.25 to 4.0) mg/kg.

2. The method of claim 1, wherein the number of the drug moiety per antibody (DAR) ranges from 1 to 7, optionally from 3 to 5 or from 3 to 6.

3. The method of claim 1, wherein the dose is 0.50, 0.75, 1.00, 1.25, 1.50, 1.75, 2.00, 2.25, 2.50, 2.75, or 3.00 mg/kg.

4. The method of claim 1, wherein the immunoconjugate is administered in three-week cycles and administered on Day 1 of each cycle at said dose.

5. The method of claim 1, wherein the immunoconjugate is administered in three-week cycles and administered on Days 1 and 8 of each cycle at said dose.

6. The method of claim 1, wherein the immunoconjugate is administered in four-week cycles and administered on Days 1, 8, and 15 of each cycle at said dose.

7. The method of claim 4, wherein the number of cycles is 3 or more.

8. The method of claim 1, wherein the immunoconjugate is administered:
a) weekly for the first three weeks and then every three weeks;
b) weekly for the first four weeks and then every three weeks;
c) weekly for the first six weeks and then every three weeks;
d) weekly for the first eight weeks and then every three weeks;
e) every three weeks for the first three weeks and then weekly;
f) every three weeks for the first six weeks and then weekly; or
g) every three weeks for the first nine weeks and then weekly.

9. The method of claim 1, wherein the immunoconjugate is administered intravenously.

10. The method of claim 1, wherein the cancer is a hematological cancer.

11. The method of claim 1, wherein the cancer is a solid tumor.

12. The method of claim 1, wherein the cancer is selected from the group consisting of chronic lymphocytic leukemia (CLL), small lymphocytic lymphoma (SLL), mantle cell lymphoma (MCL), follicular lymphoma (FL), marginal zone lymphoma (MZL), diffuse large B-cell lymphoma (DLBCL), Richter transformation lymphoma (RTL), Burkitt lymphoma (BL), lymphoplasmacytoid lymphoma (LPL), Waldenström macroglobulinemia (WM), T cell non-Hodgkin lymphoma, acute lymphocytic leukemia (ALL), and acute myeloid leukemia (AML).

13. The method of claim 1, wherein the patient has been treated previously for the cancer.

14. The method of claim 12, wherein the cancer is relapsed or refractory to treatment.

15. The method of claim 1, wherein treatment with the immunoconjugate results in one or more of the following:
a) induces tumor regression:
b) delays tumor progression;
c) inhibits cancer metastasis:
d) prevents cancer recurrence or residual disease;
e) decreases the size of nodal or extranodal tumor masses;
f) decreases malignant cell numbers in bone marrow and peripheral blood;
g) decreases malignant splenomegaly or hepatomegaly;
h) improves cancer-related anemia, neutropenia, or thrombocytopenia;
i) ameliorates cutaneous manifestation;
j) decrease the likelihood of hyperviscosity syndrome in patients with LPL/WM;
k) ameliorates disabling constitutional symptoms; and
l) prolongs survival.

16. The method of claim 1, wherein treatment with the immunoconjugate results in complete tumor eradication.

17. (canceled)

18. (canceled)
